SEQUENCE LISTING (2 sequences)

SEQ ID NO: 1 - protein, 1096 aa, Homo sapiens
MADHVQSLAQLENLCKQLYETTDTTTRLQAEKALVEFTNSPDCLSKCQLLLERGSSSYSQLLAATCLTKLVSRTNNPLPLEQRIDIRNYVLNYLATRPKLATFVTQALIQLYARITKLGWFDCQKDDYVFRNAITDVTRFLQDSVEYCIIGVTILSQLTNEINQVSATAFLIEADTTHPLTKHRKIASSFRDSSLFDIFTLSCNLLKQASGKNLNLNDESQHGLLMQLLKLTHNCLNFDFIGTSTDESSDDLCTVQIPTSWRSAFLDSSTLQLFFDLYHSIPPSFSPLVLSCLVQIASVRRSLFNNAERAKFLSHLVDGVKRILENPQSLSDPNNYHEFCRLLARLKSNYQLGELVKVENYPEVIRLIANFTVTSLQHWEFAPNSVHYLLSLWQRLAASVPYVKATEPHMLETYTPEVTKAYITSRLESVHIILRDGLEDPLEDTGLVQQQLDQLSTIGRCEYEKTCALLVQLFDQSAQSYQELLQSASASPMDIAVQEGRLTWLVYIIGAVIGGRVSFASTDEQDAMDGELVCRVLQLMNLTDSRLAQAGNEKLELAMLSFFEQFRKIYIGDQVQKSSKLYRRLSEVLGLNDETMVLSVFIGKIITNLKYWGRCEPITSKTLQLLNDLSIGYSSVRKLVKLSAVQFMLNNHTSEHFSFLGINNQSNLTDMRCRTTFYTALGRLLMVDLGEDEDQYEQFMLPLTAAFEAVAQMFSTNSFNEQEAKRTLVGLVRDLRGIAFAFNAKTSFMMLFEWIYPSYMPILQRAIELWYHDPACTTPVLKLMAELVHNRSQRLQFDVSSPNGILLFRETSKMITMYGNRILTLGEVPKDQVYALKLKGISICFSMLKAALSGSYVNFGVFRLYGDDALDNALQTFIKLLLSIPHSDLLDYPKLSQSYYSLLEVLTQDHMNFIASLEPHVIMYILSSISEGLTALDTMVCTGCCSCLDHIVTYLFKQLSRSTKKRTTPLNQESDRFLHIMQQHPEMIQQMLSTVLNIIIFEDCRNQWSMSRPLLGLILLNEKYFSDLRNSIVNSQPPEKQQAMHLCFENLMEGIERNLLTKNRDRFTQNLSAFRREVNDSMKNSTYGVNSNDMMS

SEQ ID NO: 2 - protein, 1235 aa, Homo sapiens
MSFFNFRKIFKLGSEKKKKQYEHVKRDLNPEDFWEIIGELGDGAFGKVYKAQNKETSVLAAAKVIDTKSEEELEDYMVEIDILASCDHPNIVKLLDAFYYENNLWILIEFCAGGAVDAVMLELERPLTESQIQVVCKQTLDALNYLHDNKIIHRDLKAGNILFTLDGDIKLADFGVSAKNTRTIQRRDSFIGTPYWMAPEVVMCETSKDRPYDYKADVWSLGITLIEMAEIEPPHHELNPMRVLLKIAKSEPPTLAQPSRWSSNFKDFLKKCLEKNVDARWTTSQLLQHPFVTVDSNKPIRELIAEAKAEVTEEVEDGKEEDEEEETENSLPIPASKRASSDLSIASSEEDKLSQNACILESVSEKTERSNSEDKLNSKILNEKPTTDEPEKAVEDINEHITDAQLEAMTELHDRTAVIKENEREKRPKLENLPDTEDQETVDINSVSEGKENNIMITLETNIEHNLKSEEEKDQEKQQMFENKLIKSEEIKDTILQTVDLVSQETGEKEANIQAVDSEVGLTKEDTQEKLGEDDKTQKDVISNTSDVIGTCEAADVAQKVDEDSAEDTQSNDGKEVVEVGQKLINKPMVGPEAGGTKEVPIKEIVEMNEIEEGKNKEQAINSSENIMDINEEPGTTEGEEITESSSTEEMEVRSVVADTDQKALGSEVQDASKVTTQIDKEKKEIPVSIKKEPEVTVVSQPTEPQPVLIPSININSDSGENKEEIGSLSKTETILPPESENPKENDNDSGTGSTADTSSIDLNLSISSFLSKTKDSGSISLQETRRQKKTLKKTRKFIVDGVEVSVTTSKIVTDSDSKTEELRFLRRQELRELRFLQKEEQRAQQQLNSKLQQQREQIFRRFEQEMMSKKRQYDQEIENLEKQQKQTIERLEQEHTNRLRDEAKRIKGEQEKELSKFQNMLKNRKKEVINEVEKAPKELRKELMKRRKEELAQSQHAQEQEFVQKQQQELDGSLKKIIQQQKAELANIERECLNNKQQLMRAREAAIWELEERHLQEKHQLLKQQLKDQYFMQRHQLLKRHEKETEQMQRYNQRLIEELKNRQTQERARLPKIQRSEAKTRMAMFKKSLRINSTATPDQDRDKIKQFAAQEEKRQKNERMAQHQKHENQMRDLQLQCEANVRELHQLQNEKCHLLVEHETQKLKELDEEHSQELKEWREKLRPRKKTLEEEFARKLQEQEVFFKMTGESECLNPSTQSRISKFYPIPSLHSTGS

(19) United States
(12) Patent Application Publication
(10) Pub. No.: US 2025/0339421 A1
(43) Pub. Date: Nov. 6, 2025

(54) METHODS FOR TREATING BILE DUCT CANCERS WITH TIVOZANIB

(71) Applicant: The U.S.A., as represented by the Secretary, Department of Health and Human Services, Bethesda, MD (US)

(72) Inventors: Jonathan Matthew Hernandez, Washington, DC (US); Reed Ayabe, Houston, TX (US); Tahsin Khan, Rockville, MD (US)

(73) Assignee: The U.S.A., as represented by the Secretary, Department of Health and Human Services, Bethesda, MD (US)

(21) Appl. No.: 18/713,533

(22) PCT Filed: Nov. 26, 2021
(86) PCT No.: PCT/US2021/060902

§ 371 Date: May 24, 2024

(51) Int. Cl. A61K 31/4709

(52) U.S. Cl. CPC A61K 31/4709

(57) ABSTRACT

Disclosed herein are methods directed to treating bile duct cancers, including cholangiocarcinoma, with tivozanib. The bile duct cancers may be advanced, metastatic or recurrent. The invention also includes methods of identifying subjects having bile duct cancers that express exportin 7 (XP07) or Ste-20 like kinase (SLK) and treating them with tivozanib.

### FIELD

The field of the invention is medicine, oncology, and the treatment of bile duct cancer.

### BACKGROUND

Cholangiocarcinoma is a rare malignant tumor that originates from the epithelial cells of the bile duct system. Approximately 10,000 new cases are diagnosed annually in the United States, and 5-year survival rate is below 20%. Currently, while surgical resection can be curative, cholangiocarcinoma has a high recurrence rate after resection. Further, most bile duct cancer is detected at an inoperable stage. There are limited effective chemotherapeutic regimens for advanced cholangiocarcinoma (recurrent or metastatic), but even with treatment, the prognosis is poor. Combination chemotherapy with gemcitabine and cisplatin is the most validated first-line treatment, but the response rate approaches only 22% and median progression-free survival is 8 months. However, a need exists for effective treatments for bile duct cancer, including cholangiocarcinoma.

Tivozanib (previously known as AV-951 and KRN951) is a potent and selective small-molecule inhibitor of vascular endothelial growth factor (VEGF) receptor tyrosine kinase inhibitor (VEGF TKI) that has been approved by the European Medicines Agency and U.S. Food and Drug Administration for the treatment of advanced or refractory renal cell carcinoma (RCC). In vitro cellular kinase assays demonstrated that tivozanib inhibits phosphorylation of vascular endothelial growth factor receptor (VEGFR)-1, VEGFR-2 and VEGFR-3 and inhibits other kinases including c-kit and PDGFR β at clinically relevant concentrations. In tumor xenograft models in mice and rats, tivozanib inhibited angiogenesis, vascular permeability, and tumor growth of various tumor cell types including human renal cell carcinoma. However, tivozanib has not been previously evaluated as a treatment for bile duct cancer, such as cholangiocarcinoma.

### SUMMARY

The present disclosure provides improved methods for treating subjects with bile duct cancer, including cholangiocarcinoma, with tivozanib. It also provides methods of identifying patients with bile duct cancer, including cholangiocarcinoma, who should be identified for treatment with tivozanib.

In one embodiment, the disclosure provides a method of treating bile duct cancer, including cholangiocarcinoma, in a subject in need thereof. The method includes administering an effective amount of tivozanib to the subject, thereby to treat the bile duct cancer.

In another embodiment, the disclosure provides a method of treating bile duct cancer, including cholangiocarcinoma, in a subject in need thereof. The method includes administering an effective amount of tivozanib to a subject identified as having bile duct cancer that expresses exportin 7 (XPO7) and/or STE-20 like kinase (SLK), thereby to treat the bile duct cancer. In one embodiment, if a subject is identified as having bile duct cancer that does not express XPO7 or SLK, tivozanib is not administered to the subject. In one embodiment, the subject is identified as having a bile duct cancer that expresses XPO7. In another embodiment, the XPO7 is detected in the cytoplasm of bile duct tumor cells from the subject. In one embodiment, expression of XPO7 is detected by immunohistochemical analysis of a tissue sample from the bile duct cancer using an anti-XPO7 antibody. In one embodiment, the subject is identified as having a bile duct cancer that expresses SLK. In another embodiment, the SLK is detected in the cytoplasm of bile duct tumor cells from the subject. In one embodiment, expression of SLK is detected by immunohistochemical analysis of a tissue sample from the bile duct cancer using an anti-SLK antibody. In one embodiment, the subject is identified as having a bile duct cancer that expresses SLK and XPO7.

In yet another embodiment, the disclosure provides a method of identifying a subject having a bile duct cancer who is eligible for treatment with tivozanib. The method includes determining whether the bile duct cancer expresses exportin 7 (XPO7) or STE-20 like kinase (SLK). The subject is eligible for treatment with tivozanib if the bile duct cancer expresses exportin 7 (XPO7) and/or STE-20 like kinase (SLK). According to one embodiment, the subject is not eligible for treatment with tivozanib if the bile duct cancer does not express XPO7 and/or does not express STE-20 like kinase (SLK). In one embodiment, the method further comprises administering an effective amount of tivozanib to the subject, thereby to treat the bile duct cancer. In one embodiment, the subject has a bile duct cancer that expresses XPO7. In another embodiment, the XPO7 is detected in the cytoplasm of bile duct tumor cells from the subject. In one embodiment, the subject has a bile duct cancer that expresses SLK. In another embodiment, the SLK is detected in the cytoplasm of bile duct tumor cells from the subject. In another embodiment, the subject is eligible for treatment with tivozanib if XPO7 is detected in the cytoplasm of bile duct tumor cells. In another embodiment, the subject is not eligible if expressed XPO7 is detected in the nuclei of cells of the bile duct cancer, but not in the cytoplasm. In another embodiment, the subject is eligible for treatment with tivozanib if XPO7 is detected in both the cytoplasm and the nuclei of bile duct tumor cells. In one embodiment, expression of XPO7 is detected by immunohistochemical analysis of a tissue sample from the bile duct cancer using an anti-XPO7 antibody. In one embodiment, the subject is identified as eligible for treatment with tivozanib if the bile duct tumor cells express SLK and XPO7 in the cytoplasm of the tumor cells. In one embodiment, expression of SLK is detected by immunohistochemical analysis of a tissue sample from the bile duct cancer using an anti-SLK antibody.

In yet another embodiment, the disclosure provides a method of inhibiting SLK in a bile duct cancer by administering an effective amount of tivozanib to the bile duct cancer, thereby to inhibit SLK in the cancer or tumor. In one embodiment, the bile duct cancer is in a human subject.

In some embodiments, the bile duct cancer is cholangiocarcinoma (CCA). In further embodiments, the bile duct cancer is an intrahepatic cholangiocarcinoma or extrahepatic cholangiocarcinoma.

In some embodiments, the bile duct cancer has been previously treated with chemotherapy. For example, the bile duct cancer has been previously treated with a platinum chemotherapy. In one embodiment, the bile duct cancer has been previously treated with oxaliplatin. In another embodiment, the bile duct cancer has been previously treated with carboplatin. In another embodiment, the bile duct cancer has been previously treated with cisplatin.

In some embodiments, the bile duct cancer has been previously treated with an antimetabolite. In one embodiment, the bile duct cancer has been previously treated with gemcitabine. In another embodiment, the bile duct cancer has been previously treated with capecitabine.

In some embodiments, the bile duct cancer has been previously treated with a fluoropyrimidine, such as 5-fluorouracil (5-FU).

In some embodiments, the bile duct cancer has been previously treated with cisplatin and gemcitabine.

In some embodiments, the bile duct cancer has been previously treated with an FGFR2 inhibitor. In one embodiment, the FGFR2 inhibitor is pemigatinib or infigratinib.

In some embodiments, the bile duct cancer has been previously treated with an isocitrate dehydrogenase 1 (IDH1) inhibitor. In some embodiments, the IDH1 inhibitor is ivosidenib.

In some embodiments, the bile duct cancer has been previously treated with a checkpoint inhibitor. In some embodiments, the checkpoint inhibitor is an anti-PD1, anti-PD-L1, or CTLA-4 inhibitor. In some embodiments, the checkpoint inhibitor is pembrolizumab, nivolumab, cemiplumab, atezolizumab, avelumab, durvalumab, ipilimumab, tremelimumab or tisotumab.

In some embodiments, the bile duct cancer was previously treated with radiation.

In some embodiments, the bile duct cancer was previously surgically resected and has recurred or metastasized.

In some embodiments, the bile duct cancer is unresectable.

In some embodiments, the bile duct cancer has not been previously treated by chemotherapy, immunotherapy, radiation, or other non-surgical intervention.

In some embodiments, the effective amount of tivozanib is 0.1 mg to 2.0 mg. In some embodiments, the effective amount of tivozanib is 1.0 mg to 1.5 mg. In some embodiments, the tivozanib is tivozanib hydrochloride. In some embodiments, the effective amount of tivozanib is 0.89 mg to 1.34 mg of tivozanib free base. In some embodiments, the effective amount of tivozanib is a treatment cycle of 1.5 mg tivozanib hydrochloride or 1.34 mg tivozanib free base administered once daily for 21 days followed by 7 days without administration of tivozanib. In other embodiments, the effective amount of tivozanib is a treatment cycle of 1.0 mg tivozanib hydrochloride or 0.89 mg tivozanib free base administered once daily for 21 days followed by 7 days without administration of tivozanib. In yet other embodiments, the effective amount of tivozanib is a treatment cycle of 0.89 mg tivozanib free base administered every other day for 28 days. In yet other embodiments, the effective amount of tivozanib is a treatment cycle of 1.34 mg tivozanib free base administered every other day for 28 days. In some embodiments, the tivozanib is administered orally. In some embodiments, the tivozanib is a capsule or tablet.

In some embodiments, the tivozanib treatment cycle is repeated 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more times. In some embodiments, the tivozanib treatment cycle is repeated until the bile duct cancer progresses, the subject dies, or the subject experiences an unacceptable toxicity.

In some embodiments, the bile duct cancer is advanced, recurrent, or metastatic bile duct cancer.

These and other aspects and features of the disclosure are described in the following detailed description and claims.

### BRIEF DESCRIPTION OF THE DRAWINGS

FIGS. 1A-1E are a series of images and plots showing that cytoplasmic exportin 7 (XPO7) is a prognostic marker for poor clinical outcomes in cholangiocarcinoma.

FIG. 1A shows exemplary images of benign bile duct tissue and cholangiocarcinoma (CCA) tissue subjected to immunohistochemical staining for expression of XPO7.

FIG. 1B is a pair of pie charts showing XPO7 expression in benign patients (left pie chart) versus CCA patients (right pie chart). In benign patients there was no evidence of XPO7 expression in the cytoplasm of bile duct cells (56% percent of benign patients expressed XPO7 in the nucleus and 44% of benign patients had no XPO7 expression). In the CCA patients, 56% had XPO7 expressed in the cytoplasm; 39% had no XPO7 expression; and 6% had nuclear expression only.

FIGS. 1C-D show Kaplan-Meier survival curves demonstrating reduced survival in XPO7 cytoplasmic-expressing patients (“XPO7+”) in two independent patient cohorts as compared to patients that did not have cytoplasmic XPO7 expression (“XPO7-”). FIG. 1C is based on a cohort of 60 subjects stratified by XPO7 immunohistochemical (IHC) staining from whole tissue, whereas FIG. 1D is based on a using tumor microarray data from 161 CCA patients.

FIG. 1E is a graph showing a multivariate analysis providing hazard ratios for various characteristics of patients with CCA. The data shows that cytoplasmic XPO7 expression is a marker of poor prognosis. The ratios with error bars for pT stage, XPO7 cytoplasm positive and lymph node metastases (in red) are to the right of the vertical line (ratio 1.0), indicating these features are indicative of a poor prognosis of CCA patients. The error bars for sex, moderate/poor differentiation and age >60 (in black) cross to the left of the vertical line (ratio 1.0).

FIGS. 2A-2I are a series of images and plots showing XPO7 is an oncogenic driver in cholangiocarcinoma and interacts with the serine-threonine kinase STE20 Like Kinase (SLK).

FIG. 2A shows that WITT cells with XPO7 knockdown formed fewer tumor organoids compared to WITT cells subject to scramble (control).

FIG. 2B shows photographs of WITT and EGI-1 cell line tumors grown in vivo in a subcutaneous murine xenograft model subjected to scramble (control) or XPO7 knockdown. The tumors (white area) formed in the XPO7 knockdowns are smaller than in the scramble (control). This is further shown in the graphs in FIG. 2C (EGI-1; p=0.001) and FIG. 2D (WITT cell line; p=0.040), which demonstrate that the tumor size in the XPO7 knockdown is statistically significantly smaller than in the control. FIGS. 2A-2D therefore show that XPO7 knockdown abrogates tumor organoid formation and in vivo growth in the extrahepatic cholangiocarcinoma cell lines EGI-1 and WITT.

FIG. 2E provides images of the tumors from the murine xenograft models in the EGI-1 (left) and WITT (right) cell lines subject to scramble (Top) and XPO7 knockdown (bottom), showing that XPO7 knockdown produces hypocellular and cystic-appearing tumors, as compared to the control. The cystic tumors are well circumscribed, have a more benign appearance and lack the associated parenchymal destruction seen in the controls. While some tumor cells remain in the cystic tumors, they are behaving less aggressively compared to the control.

FIG. 2F is a Venn-diagram showing results of an immunoprecipitation-mass spectrometry experiment from cholangiocarcinoma cell lines EGI-1 and WITT. In this study, immunoprecipitation experiments were performed on the cytoplasm of bile duct cancer cell lines using anti-XPO7 antibody. The precipitated proteins were evaluated by mass spectrometry. SLK was the most abundant protein identified, indicating SLK is a binding partner for XPO7.

FIG. 2G is an immuno-electron microscopy image confirming the XPO7-SLK interaction in the cytoplasm of CCA tumor cells. Circles labeled “+” indicate SLK in the cytoplasm, whereas the other circles indicate XPO7. The “+” labeled SLK are interacting with XPO7.

FIG. 2H provides images of tissues from a murine xenograft model using EGI-1 and WITT CCA cell lines subjected either to scramble (control) or knockdown of SLK. The cell lines subjected to the control (scramble) had visible whitish tumor masses, whereas those subjected to SLK knockdown had much smaller tumor masses, showing SLK knockdown abrogates tumor growth in vivo.

FIG. 2I is a bar graph showing normalized phospho-protein levels in control and SLK knockdowns of CCA tissue (EGI-1 and WITT) resulting from a phosphokinase array. Control values are indicated by the left bar and SLK knockdown values are indicated by the right bar for each phosphoprotein assayed. The results indicate a robust decrease in AKT S473 phosphorylation upon SLK knockdown.

FIG. 2J is a Western blot showing XPO7 and SLK knockdown decreased AKT S473 phosphorylation in EGI-1 and WITT CCA cell lines.

FIGS. 3A-3K are a series of plots and images showing use of an SLK small molecule inhibitor as a therapeutic strategy to treat cholangiocarcinoma.

FIG. 3A is shows a kinome inhibition profile of tivozanib which revealed inhibitory activity against SLK.

FIG. 3B is a bar graph showing tivozanib inhibits AKT S473 phosphorylation in three cholangiocarcinoma cell lines (WITT, EGI-1, and SNU-1079) compared to control levels.

FIGS. 3C-3E are scatter plots showing IC50 values for tivozanib in three cholangiocarcinoma cell lines (WITT, EGI-1, and SNU-1079), which show that tivozanib inhibits proliferation of all three of these cholangiocarcinoma cell lines.

FIG. 3F shows images demonstrating that tivozanib abrogates tumor organoid formation in three cholangiocarcinoma cell lines (WITT, EGI-1, and SNU-1079). Images of control are on the top row, whereas the effects of tivozanib at 200 nM are shown in the bottom row where the tumor organoids are smaller and fewer than in the control.

FIG. 3G is a photograph of tumors obtained in a subcutaneous xenograft model using athymic nude mice (top row=vehicle treated; bottom row=20 mg/kg/day Tivozanib treatment for 28 days).

FIG. 3H shows the quantification of final tumor volumes from the vehicle- and tivozanib-treated tumors shown in FIG. 3F. The tumor volumes of the tivozanib treated tumors were significantly smaller than in the control (p value of <0.001).

FIG. 3I is a waterfall plot showing substantial change in tumor volume over the course of Tivozanib treatment.

FIG. 3J shows micrographs of cholangiocarcinoma obtained from a patient and stained with hematoxylin and eosin (H&E) (top row) and Ki67 (bottom row). By day 4, the tivozanib treated tumor tissue showed reduction in dark stained nuclei (bottom row) as well as increased degeneration and necrosis, whereas on day 4, control tissue (0 μg/mL tivozanib) showed dark stained nuclei (bottom row) and histopathological indications of cancer.

FIG. 3K shows the percentage of cells that are viable (left bar), degenerate (middle bar) or necrotic (right bar) for each of the control, 0.2 μg/mL tivozanib treated CCA, and 1.0 μg/mL tivozanib treated tumors shown in FIG. 3J. The data demonstrate that tivozanib treatment resulted in decreased viability and increased degeneration and necrosis compared to the control.

FIG. 3L provides a photograph of tumors obtained in an EGI-1 CCA cell line subcutaneous xenograft model following 28 days of treatment with vehicle or bevacizumab (top row=vehicle treated; bottom row=bevacizumab). Bevacizumab was provided at the standard murine dose. As the accompanying bar graph shows, the average tumor volume for bevacizumab treatment was lower than the control. However, the reduction was not as dramatic as that seen with tivozanib alone (see FIG. 3H). As bevacizumab is an anti-VEGF antibody, the reduction seen in tumor volume provided by tivozanib cannot be attributed to its VEGF inhibitory properties alone; otherwise, a similar reduction would have been expected with bevacizumab.

FIG. 4 is an exemplary human XPO7 protein sequence (GenBank Accession No. NP_001093631.1)

FIG. 5 is an exemplary human SLK protein sequence (GenBank Accession No. NP_055535.2).

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file, created on Nov. 26, 2021, 19.9 KB, which is incorporated by reference herein. In the accompanying sequence listing:

SEQ ID NO: 1 is an exemplary amino acid sequence of human XPO7.

SEQ ID NO: 2 is an exemplary amino acid sequence of human SLK.

### DETAILED DESCRIPTION

The present disclosure provides methods of treating bile duct cancer using tivozanib, as well as methods of identifying subjects having bile duct cancer to treat with tivozanib.

The disclosed subject matter is based, in part, on the discovery, reported herein, of the interaction between the human nuclear export protein exportin 7 (XPO7) and a hitherto incompletely studied kinase, Ste-20 like kinase (SLK). The studies disclosed herein demonstrate that cytoplasmic accumulation of XPO7 is predictive of poor outcomes for patients with bile duct cancer, such as cholangiocarcinoma (CCA). The Examples provided herein show that XPO7 is an oncogenic driver in CCA cells and binds to and promotes cytoplasmic localization and stabilization of SLK, which in turn activates oncogenic AKT signaling, establishing SLK as a novel, bona fide target in cholangiocarcinoma and other bile duct cancers.

The disclosed subject matter is also based, in part, on the discovery, reported herein, that the pan-vascular endothelial growth factor receptor (VEGFR) inhibitor tivozanib, has activity against SLK, reducing AKT phosphorylation and abrogating growth of CCA tumor organoids, and, in a murine xenograft model and an ex vivo tumor platform using a liver metastasis from a patient with XPO7-expressing cholangiocarcinoma, was effective in inducing tumor cell degradation and death.

As demonstrated herein, tivozanib is useful in treating patients with bile duct cancers, such as cholangiocarcinoma. Tivozanib is also useful in treating patients whose bile duct cancers express XPO7, particularly in the cytoplasm of tumor cells. Patients identified as having bile duct cancers that express XPO7, particularly in the cytoplasm of tumor cells can be selected for treatment with tivozanib. SLK is another marker that is useful in determining or identifying patients having bile duct cancer that can be treated with tivozanib. Tivozanib can be used to decrease or inhibit the activity of SLK in bile duct tumor cells both in vitro, and in vivo, for example in human subjects suffering from bile duct cancer.

### I. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. For example, the term “a cell” includes single or plural cells and can be considered equivalent to the phrase “at least one cell.” Similarly, the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context. Further, elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure(s). For example, it will be appreciated that all features described and depicted herein can be applicable to all aspects of the disclosure(s) described and depicted herein.

As used herein, the term “comprises” means “includes.” The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. All publications, patent applications, patents, GenBank accession numbers and other references mentioned herein are incorporated by reference in their entirety.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

The order of steps or order for performing certain actions is immaterial so long as the present disclosure remain operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the description unless claimed. No language in the specification should be construed as indicating any non-claimed element as essential to the practice of the present disclosure.

For convenience, certain terms in the specification, examples, and appended claims are collected in this section.

Administration: To provide or give a subject an agent, such as tivozanib, by any effective route. Exemplary routes of administration include, but are not limited to, oral, injection (such as subcutaneous, intramuscular, intradermal, intraperitoneal, intravenous, intra-arterial (including hepatic intra-arterial), intra-ductal, intraprostatic, and intratumoral), sublingual, rectal, transdermal, intranasal, vaginal and inhalation routes. In some examples, administration is local. In other examples, administration is systemic.

Advanced (cancer or tumor): A cancer or tumor, i.e., bile duct cancer, e.g., cholangiocarcinoma, that has reached Stage 3 or Stage 4. In certain embodiments, “advanced” means that the cancer or tumor has metastasized, or otherwise cannot be adequately treated with local therapy alone, such as surgical intervention or radiation therapy, and therefore requires a systemic therapy. In certain embodiments, “advanced” means that the cancer or tumor has recurred after having previously responded to treatment with a local or systemic therapy. In certain embodiments, advanced cancer is unlikely to be cured or controlled with treatment and, therefore, in a subject with advanced bile duct cancer, palliative therapy is used to slow the growth of the cancer or to relieve symptoms through end of life. In some contexts, advanced cancer includes recurrent cancer, e.g., recurrent bile duct cancer.

Bile duct cancer: Any cancer or tumor that forms in the bile ducts, including cholangiocarcinoma. The vast majority of bile duct cancers are cholangiocarcinomas, which are adenocarcinomas forming in the gland cells (epithelial cells) of the bile duct system; however, bile duct sarcomas, lymphomas, and small cell cancers can occur. Bile duct cancers, including cholangiocarcinoma, are defined by the location where they start growing. For example, cancers that form in the bile ducts in the liver are classified as intrahepatic bile duct cancers, while cancers that form in the bile ducts outside the liver are classified as extrahepatic bile duct cancers. For example, intrahepatic cholangiocarcinoma can be found in liver parenchyma. Extrahepatic bile duct cancers, including cholangiocarcinomas, include (1) perihilar or hilar bile duct cancers forming within the perihilar bile ducts, also known as Klatskin tumors, and (2) distal bile duct cancers that form in the distal bile ducts. Extrahepatic cholangiocarcinoma is the most common form of bile duct cancer, with Klatskin tumors of the perihilar ducts being the most common type of cholangiocarcinoma. In some embodiments, the methods disclosed herein can be used to treat the aforementioned types of bile duct cancers.

Bile Duct Cancer that expresses exportin 7 (XPO7) protein in the cytoplasm: Any bile duct cancer, including cholangiocarcinoma, in which bile duct tumor cells exhibit cytoplasmic XPO7, a protein found in the nucleus of normal cells. Bile duct cancers that express XPO7 in the cytoplasm can be identified, for example, by immunoassay using an antibody that specifically binds XPO7. In one example, an immunoblot (e.g., Western blot) is performed on both cytoplasmic and nuclear extracts of a bile duct tumor cell to determine localization of XPO7. In another example, microscopy (such as immunofluorescence microscopy or electron microscopy) is used to visualize localization of XPO7 in the cytoplasm or nucleus of bile duct tumor cells. Antibodies specific for XPO7 are commercially available, such as from Abcam (ab96525), Creative Diagnostics (DPABH-19601), ThermoFisher Scientific (PA5-18241, PA5-21423, PA5-111187, PA5-111268, A305-805A-M), Atlas Antibodies (HPA048153), Proteintech (12980-1-AP) and Santa Cruz Biotechnology (sc-390025). In one example, IHC is used, and the cytoplasmic staining pattern can be scored positive for weak intensity if at least 20% of the cells of the tumor had detectable cytoplasmic staining of XPO7, or for any moderate or strong cytoplasmic staining of XPO7. Thus, in some examples, a bile duct cancer that expresses cytoplasmic XPO7 (and is one that can be treated with tivozanib) is one wherein at least 20% of the cells have detectable XPO7 staining, such as at least 30%, at least 40%, at least 50%, at least 60%, at least 70%, at least 80%, at least 90%, at least 95%, at least 99%, or 100% of the cells have detectable XPO7 staining (for example in the cytoplasm). In one example, a cancer is considered positive for cytoplasmic expression of XPO7 if the sample has an H-score of 51 or greater, 101 or greater, 126 or greater, 150 or greater, or 200 or greater. In one example staining intensity is scored as weak (1), moderate (2), or strong (3). In another example, immunoprecipitation is used, and the bile duct cancer is determined to be one that expresses XPO7 cytoplasmically if there is at least 20% more detectable XPO7 in the cytoplasm fraction as compared to the nuclear fraction. Thus, in some examples, a bile duct cancer that expresses cytoplasmic XPO7 (and is one that can be treated with tivozanib) is one wherein there is at least 20% more detectable XPO7 in the cytoplasm fraction as compared to the nuclear fraction, such as at least 30%, at least 40%, at least 50%, at least 60%, at least 70%, at least 80%, at least 90%, at least 95%, at least 99%, at least 100%, at least 200%, at least 300%, at least 400%, or at least 500% more detectable XPO7 in the cytoplasm fraction as compared to the nuclear fraction. Similarly, a bile duct cancer that expresses SLK protein in the cytoplasm is any type of bile duct cancer in which the tumor cells exhibit cytoplasmic SLK, a protein found in the nucleus of normal cells. Bile duct cancers that express SLK in the cytoplasm can be identified, for example, by immunoassay using an antibody that specifically binds SLK. In one example, a Western blot is performed on both cytoplasmic and nuclear extracts of a tumor cell to determine localization of SLK. In another example, microscopy (such as immunofluorescence microscopy or electron microscopy) is used to visualize localization of SLK in the cytoplasm or nucleus of bile duct tumor cells. Antibodies specific for SLK are commercially available, such as from Cell Signaling Technology (Catalog #41255), Abcam (Catalog #ab65113, ab226986 and ab70230), LSBio (Catalog LS-C752943), Santa Cruz Biotechnology (Catalog sc-515493), and Novus Biologicals (Catalog H00009748-M01, NBP2-20401, NBP1-83024, NBP3-04553, NBP2-98482, and NBP2-98859). In one example, IHC is used, and the cytoplasmic staining pattern can be scored positive for weak intensity if at least 20% of the cells of the tumor had detectable cytoplasmic staining of SLK, or for any moderate or strong cytoplasmic staining of SLK. Thus, in some examples, a bile duct cancer that expresses cytoplasmic SLK (and is one that can be treated with tivozanib) is one wherein at least 20% of the cells have detectable SLK staining, such as at least 30%, at least 40%, at least 50%, at least 60%, at least 70%, at least 80%, at least 90%, at least 95%, at least 99%, or 100% of the cells have detectable SLK staining (for example in the cytoplasm). In one example, a cancer is considered positive for cytoplasmic expression of SLK if the sample has an H-score of 51 or greater, 101 or greater, 126 or greater, 150 or greater, or 200 or greater. In one example staining intensity is scored as weak (1), moderate (2), or strong (3). In another example, immunoprecipitation is used, and the bile duct cancer is determined to be one that expresses SLK cytoplasmically if there is at least 20% more detectable SLK in the cytoplasm fraction as compared to the nuclear fraction. Thus, in some examples, a bile duct cancer that expresses cytoplasmic SLK (and is one that can be treated with tivozanib) is one wherein there is at least 20% more detectable SLK in the cytoplasm fraction as compared to the nuclear fraction, such as at least 30%, at least 40%, at least 50%, at least 60%, at least 70%, at least 80%, at least 90%, at least 95%, at least 99%, at least 100%, at least 200%, at least 300%, at least 400%, or at least 500% more detectable SLK in the cytoplasm fraction as compared to the nuclear fraction. In some examples, a bile duct cancer is a bile duct cancer that expresses both XPO7 and SLK protein in the cytoplasm, and combinations of these results are observed in the bile duct cancer.

Chemotherapeutic agent/Chemotherapy: Any chemical or biological agent with therapeutic usefulness in the treatment of diseases characterized by abnormal cell growth, such as tumors, neoplasms, and cancer. These agents or drugs are categorized by their mode of activity within a cell, for example, whether and at what stage they affect the cell cycle. In one embodiment, a chemotherapeutic agent is an agent for use in treating a cytoplasmic XPO7-positive bile duct tumor (e.g., a cytoplasmic XPO7-positive cholangiocarcinoma). In one embodiment, a chemotherapeutic agent is a radioactive compound. Exemplary chemotherapeutic agents that can be used with the methods provided herein are disclosed in Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., @ 2000 Churchill Livingstone, Inc; Baltzer, L., Berkery, R. (eds.): Oncology Pocket Guide to Chemotherapy, 2nd ed. St. Louis, Mosby-Year Book, 1995; Fischer, D. S., Knobf, M. F., Durivage, H. J. (eds): The Cancer Chemotherapy Handbook, 4th ed. St. Louis, Mosby-Year Book, 1993). Examples of chemotherapeutic agents include: alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DFMO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

Cholangiocarcinoma: A type of cancer that forms in the bile ducts. Intrahepatic cholangiocarcinoma begins in the small bile ducts within the liver. Extrahepatic cholangiocarcinoma, including perihilar cholangiocarcinoma (also known as Klatskin tumor) and distal cholangiocarcinoma, forms in bile ducts outside of the liver. The most common form of cholangiocarcinoma is perihilar cholangiocarcinoma, accounting for more than 50% of all cases. Cholangiocarcinoma is typically not diagnosed until it has metastasized. Symptoms (such as jaundice) can occur when the bile ducts are blocked by tumor tissue. Other symptoms can include extreme fatigue, itching, dark-colored urine, loss of appetite, unintentional weight loss, abdominal pain, and light-colored and greasy stools.

Clinical benefit: Refers to a subject experiencing one or more of: (a) slowing of tumor growth, (b) halting of tumor growth, (c) tumor regression or disappearance, (d) amelioration of a symptom of the cancer, (e) curing the cancer, and (f) prolonging survival of the subject.

Disease control rate (DCR): The percentage of subjects with advanced or metastatic cancer, e.g., bile duct cancer such as cholangiocarcinoma, who achieve complete response (CR), partial response (PR) or stable disease (SD) in response to a cancer treatment, such as tivozanib.

Drug related adverse event: Refers to an adverse event (AE) as defined and classified in the National Cancer Institute-Common Terminology Criteria for Adverse Events (CTCAE), version 4.03, dated Jun. 14, 2010. Any reference to “Grade” of adverse event refers to the grading system as outlined therein.

Effective amount: A quantity of a specific substance (such as tivozanib) sufficient to achieve a desired effect in a subject being treated. For instance, this can be the amount necessary to inhibit or suppress growth of a tumor. In one embodiment, an effective amount is the amount necessary to eliminate, reduce the size, or prevent metastasis of a tumor, such as reduce a tumor size, weight and/or volume by at least 10%, at least 20%, at least 50%, at least 75%, at least 80%, at least 90%, at least 95%, or even 100%, and/or reduce the number and/or size/volume/weight of metastases by at least 10%, at least 20%, at least 50%, at least 75%, at least 80%, at least 90%, at least 95%, or even 100%, for example as compared to a size/volume/number/weight prior to treatment. In some examples, the methods increase the survival time of a treated subject by at least 3 months, at least 6 months, at least 9 months, at least 12 months, at least 18 months, at least 24 months, at least 36 months, at least 48 months, or at least 60 months, for example relative to the survival time in an absence of the treatment provided herein. In some examples, combinations of these effects are achieved.

When administered to a subject, a dosage will generally be used that will achieve target tissue concentrations (for example, in tumors) that has been shown to achieve a desired in vitro effect. An effective amount can be administered in one or more administrations, applications or dosages and is not limited to a particular formulation or administration route.

In some embodiments, an effective amount of tivozanib includes an effective treatment regimen of tivozanib. An “effective treatment regimen of tivozanib” refers to a treatment regimen of tivozanib sufficient to effect beneficial or desired results, such as to effect a clinical benefit in a subject. An effective treatment regimen of tivozanib can be administered in one or more administrations, applications or dosages and is not limited to a particular formulation or administration route. An exemplary effective treatment regimen of tivozanib is a regimen that is effective to elicit a complete or partial response according to RECIST (Version 1.1) criteria, as further defined herein. An exemplary effective treatment regimen of tivozanib is administration of at least one treatment cycle with tivozanib, where a treatment cycle comprises administering 1.5 mg tivozanib for 21 days followed by 7 days without administration of tivozanib. Another exemplary effective treatment regimen of tivozanib is administration of at least one treatment cycle with tivozanib, where a treatment cycle comprises administering 1.0 mg tivozanib for 21 days followed by 7 days without administration of tivozanib. Another exemplary effective treatment regimen of tivozanib is administration of at least one treatment cycle with tivozanib, where a treatment cycle comprises administering 1.0 mg tivozanib for 28 days. Another exemplary effective treatment regimen of tivozanib is administration of at least one treatment cycle with tivozanib, where a treatment cycle comprises administering 1.0 mg tivozanib every other day for 28 days. Another exemplary effective treatment regimen of tivozanib is administration of at least one treatment cycle with tivozanib, where a treatment cycle comprises administering 1.5 mg tivozanib every other day for 28 days.

Exportin 7 (XPO7): A protein involved in the transport of cargo proteins through nuclear pore complexes (Aksu et al., J Cell Biol 217 (7): 2329-2340, 2017; see also NCBI Gene ID 23039). In normal (non-tumor) cells, XPO7 protein is found in the nucleus. However, as disclosed herein, in a subset of bile duct tumor cells, such as in a subset of cells from cholangiocarcinoma, XPO7 protein is localized to the cytoplasm. Nucleic acid and protein sequences of human XPO7 are publicly available, such as those deposited under GenBank Accession Nos. NM_001100161.2 (isoform a mRNA), NP_001093631.1 (isoform a protein), NM_001362802.2 (isoform d mRNA), NP_001349731.1 (isoform d protein), NM_015024.5 (isoform b mRNA) and NP_055839.3 (isoform b protein), which are herein incorporated by reference. Homologs of human XPO7 can be found in several mammalian species, including for example, chimpanzee, Rhesus monkey, dog, cow, mouse and rat. XPO7 is also known as EXP7 and RANBP16.

Immunoassay: A biochemical test that measures the presence or concentration of a macromolecule or a small molecule, such as a protein (e.g., XPO7 or SLK), for example through the use of an antibody (which in some examples is labeled, for example with a fluorophore, enzyme, or other detectable agent) or an antigen. Examples of immunoassays include immunostaining (e.g., immunohistochemistry (IHC)) and immunoprecipitation. In one example, IHC is used to detect the presence and localization pattern of a protein (e.g., XPO7 or SLK) in a tissue sample, such as bile duct tumor tissue. Such IHC methods can include contacting the sample with an antibody, such as a XPO7-specific antibody and/or SLK-specific antibody. The antibody may be directly labeled, or a labeled secondary label can be used. Exemplary labels include fluorophores (e.g., fluorescein or rhodamine), chemiluminescent agents, enzymes (e.g., alkaline phosphatase or horseradish peroxidase), and metals (e.g., gold particles for electron microscopy), and visualized using microscopy, allowing for a determination of whether the protein is present, and its localization pattern (e.g., nucleus or cytoplasm). In one example, immunoprecipitation is used to detect the presence and localization pattern of a protein (e.g., XPO7 or SLK) in a tissue sample, such as bile duct tumor tissue.

Express/Expresses/Expressing/Expression: In the context of the proteins XPO7 or SLK, these terms refer to one or both of these proteins being present, e.g., in a cell, e.g., in the cytoplasm of a cell, e.g., a bile duct cell such as a tumor cell. For example, if XPO7 is expressed in a tumor or cancer of the bile duct, it is detectable in a cell in that tumor, such as the cytoplasm of the bile duct tumor. For example, if SLK is expressed in a tumor or cancer of the bile duct, it is detectable in a cell in that tumor, such as the cytoplasm of the bile duct tumor. Expression is detectable by means known in the art. In some embodiments, whether a protein, such as XPO7 or SLK is expressed by a bile duct tumor is determined by a trained pathologist or oncologist using clinically relevant methods, such immunohistochemical (IHC) methods, as described herein and known in the art. In some embodiments, XPO7 or SLK is expressed in a tumor or cancer of the bile duct if its level in cells from the tumor, such as in the cytoplasm, is increased compared to a cells in benign tissue or non-diseased cells, such as bile duct tissue cells. In some embodiments, XPO7 is considered detectable or a tumor or cancer of the bile duct is considered positive for XPO7 expression or is considered to express XPO7 if cytoplasmic immunostaining, e.g., with an antibody to XPO7, of a tissue section, e.g., a formalin-fixed paraffin-embedded tumor block, results in staining of at least 20% of the tumor tissue section. In some embodiments, SLK is considered detectable or a tumor or cancer of the bile duct is considered positive for XPO7 expression or is considered to express SLK if cytoplasmic immunostaining, e.g., with an antibody to XPO7, of a tissue section, e.g., a formalin-fixed paraffin-embedded tumor block, results in staining of at least 20% of the tumor tissue section.

Immunotherapy: Substances or drugs used to stimulate or suppress the immune system to treat cancer. Immunotherapy can include certain cytokines, vaccines, BCG, and certain monoclonal antibody therapies. Immunotherapy includes checkpoint inhibitors such as a PD-1 inhibitor, a PD-L1 inhibitor, a CTLA-4 inhibitor, a TIM-3 inhibitor, a LAG-3 inhibitor, a TIGIT inhibitor, a VISTA inhibitor, a KIR inhibitor, a 2B4 inhibitor, a CD160 inhibitor, a CGEN-15049 inhibitor, a CHK1 inhibitor, a CHK2 inhibitor, a A2aR inhibitor, or any combination thereof. In some embodiments, a PD-1 inhibitor includes nivolumab, pembrolizumab, pidilizumab, REGN2810, PDR001, or any combination thereof. In some embodiments, a PD-L1 inhibitor includes durvalumab, atezolizumab, avelumab, or any combination thereof. In some embodiments, a CTLA-4 inhibitor includes ipilimumab, tremelimumab, AGEN-1884, or any combination thereof. In some embodiments, a TIM-3 inhibitor includes TSR-022, LY3321367, MBG453, or any combination thereof. In some embodiments, a TIGIT inhibitor includes BMS-986207, AGEN17, tiragolumab, MK-7684, OMP-313M32, EOS-448, AB154, or combinations thereof. In some embodiments, a LAG-3 inhibitor includes BMS-986016, REGN3767, IMP321, LAG525, BI754111, favezelimab, or combinations thereof. In some embodiments, a VISTA inhibitor includes CI-8993, HMBD-002, a PSGL-1 antagonist as described in WO 2018/132476, or combinations thereof.

Metastatic cancer: A cancer (e.g., bile duct cancer) that has spread from the part of the body where it started (the primary site) to another part of the body. For example, metastatic bile duct cancer refers to primary site tumors that have spread from the bile duct to other parts of the body. In some embodiments, metastatic bile duct cancer requires palliative therapy. In bile duct cancer, liver and lymph nodes are common sites for metastasis, along with the gallbladder, pancreas, or small intestine and sites in the peritoneum of the abdomen or the small intestine (duodenum).

Objective response rate (ORR): The proportion of subjects with confirmed complete response (CR) or confirmed partial response (PR) according to response evaluation criteria in solid tumors (RECIST; Version 1.1), relative to the total population of randomized subjects. Confirmed responses are those that persist on repeat imaging study at least 4 weeks after the initial documentation of response.

Overall survival (OS): The time from the date of randomization to date of death due to any cause. In the absence of confirmation of death, survival time is censored at the last date the subject is known to be alive. Subjects lacking data beyond randomization have their survival times censored on the date of randomization.

Palliative therapy: A type of therapy that is not expected to cure a patient or a subject's cancer (such as bile duct cancer), usually because the cancer has progressed too far, is too advanced and/or has become metastatic. In such cases, palliative therapy is intended primarily to ameliorate one or more symptoms of the patient's cancer and/or to reduce the suffering caused by the cancer.

Pharmaceutical composition: The combination of an active agent (such as tivozanib) with a carrier (inert or active) that is suitable for diagnostic or therapeutic use in vivo or ex vivo.

Pharmaceutically acceptable carriers: The pharmaceutically acceptable carriers of use are conventional. Remington: The Science and Practice of Pharmacy, 22″d ed., London, UK: Pharmaceutical Press, 2013, describes compositions and formulations suitable for pharmaceutical delivery of the agents and compositions disclosed herein (such as tivozanib and pharmaceutical compositions thereof). In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop, such as a reduction in tumor burden or a decrease in the number or size of metastases.

“Ameliorating” refers to the reduction in the number or severity of signs or symptoms of a disease, such as cancer.

Recurrent cancer: Cancer (e.g., a bile duct cancer) that fails to respond to treatment or returns after treatment (the cancer “recurs”). For example, a bile duct cancer is recurrent if it fails to respond to a mode of treatment, e.g., the subject experiences disease progression while undergoing treatment. As another example, a bile duct cancer is recurrent if it returns or progresses after treatment or surgical resection. Recurrent cancer may also be a cancer that responds to an initial treatment, and then returns, or is a cancer that initially responds to a treatment, but later in the treatment process stops responding to such treatment. In certain embodiments, “recurrent” refers to a cancer or tumor, such as bile duct cancer, that has been previously treated with at least one systemic or local treatment, and has not responded to such treatment or becomes resistant to such treatment, or that continues to progress during or after such treatment.

Response: As used herein, the terms “response” or “responding” in the context of a subject's response to a therapeutic agent (such as tivozanib) refer to the RECIST (Response Evaluation Criteria in Solid Tumors, version 1.1, 2009) criteria for evaluating response of target lesions to a cancer therapy. According to the RECIST criteria, subjects who respond are categorized as either “complete responders” (disappearance of all target lesions) or “partial responders” (at least a 30% decrease in the sum of the diameters of the target lesions, taking as reference the baseline sum of diameters). Non-responders are placed into one of two categories: stable disease (SD; neither sufficient shrinkage to qualify for partial response nor sufficient increase to qualify for progressive disease, taking as reference the smallest sum diameters while on treatment) or progressive disease (PD; at least a 20% increase in the sum of the diameters of the target lesions, taking as reference the smallest sum (this includes the baseline sum if that is the smallest on study; in addition to the relative increase of 20%, the sum must also demonstrate an absolute increase of at least 5 mm; the appearance of one or more new lesions also qualifies as progression). The RECIST criteria are discussed in detail in, e.g., Therasse et al., J. NATL. CANCER INST., 2000:92:205-216 (RECIST 1.0), and Eisenhauer et al., EUR. J. CANCER, 2009:25:228-247 (RECIST 1.1). Accordingly, as described herein, responding to therapy refers to subjects falling within the RECIST categories of complete or partial responder, whereas not responding refers to subjects falling within the RECIST categories of stable disease or progressive disease.

Sample (or biological sample): A biological specimen containing genomic DNA, RNA (including mRNA), protein, or combinations thereof, obtained from a subject. Examples include, but are not limited to, peripheral blood, tissue, cells, urine, saliva, tissue biopsy, fine needle aspirate, surgical specimen, and autopsy material. In one example, a sample includes a tumor biopsy, such as a tumor tissue biopsy, e.g., from a bile duct tumor, such as cholangiocarcinoma.

Subject: Living multi-cellular vertebrate organisms, a category that includes both human and veterinary subjects, including human and non-human mammals such as pigs, mice, rats, guinea pigs, rabbits, sheep, horses, cows, dogs, cats and non-human primates (such as monkeys, chimpanzees, baboons, and rhesus macaques). As used herein, the terms “subject” and “patient” are used interchangeably. In some examples, the subject is a human.

STE20-like kinase (SLK): A serine-threonine kinase in the STE family. The SLK protein is ubiquitously expressed, with the highest levels of expression found in the heart and skeletal muscle. SLK is activated by homodimerization and autophosphorylation. SLK has been implicated in apoptosis, actin stress fiber dissolution and cancer cell motility (see, e.g., Al-Zahrani et al., Cell Adh Migr 7 (1): 1-10, 2013; UniProt Q9H2G2; and NCBI Gene ID 9748). There are two isoforms of SLK, produced by alternative splicing. Isoform 1 is the longer variant; isoform 2 lacks an in-frame exon in the 3′ coding region. Nucleic acid and protein sequences of human SLK are publicly available, such as those deposited under GenBank Accession Nos. NM_001304743.2 (isoform 2 mRNA), NP_001291672.1 (isoform 2 protein), NM_014720.4 (isoform 1 mRNA), and NP_055535.2 (isoform 1 protein), which are herein incorporated by reference. Homologs of human SLK can be found in several mammalian species, including for example, chimpanzee, Rhesus monkey, dog, cow, mouse and rat. Detecting expression of SLK protein can be performed, for example, by immunoassay using an antibody that specifically binds SLK. SLK-specific antibodies are commercially available, such as from Cell Signaling Technology (Catalog #41255), Abcam (Catalog #ab65113, ab226986 and ab70230), LSBio (Catalog LS-C752943), Santa Cruz Biotechnology (Catalog sc-515493), and Novus Biologicals (Catalog H00009748-M01, NBP2-20401, NBP1-83024, NBP3-04553, NBP2-98482, and NBP2-98859).

Tivozanib: A small molecule having the chemical name N-{2-chloro-4-[(6,7-dimethoxy-4-quinolyl)oxy]-phenyl}-N′-(5-methyl-3-isoxazolyl) urea and having the following chemical structure:

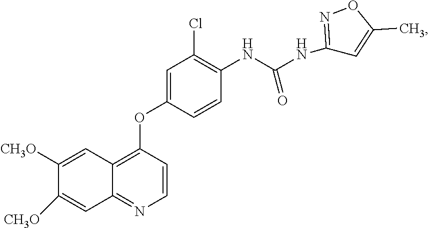

including pharmaceutically acceptable salts, solvates, solvates of a pharmaceutically acceptable salt, esters, or polymorphs thereof. See, for example, U.S. Pat. Nos. 6,821,987, 7,166,722 and 7,211,587, each of which are incorporated herein by reference in their entirety. Tivozanib is sold under the tradename FOTIVDA® in the United States by Aveo Pharmaceuticals, Inc. (Cambridge, MA). In certain embodiments, tivozanib is N-{2-chloro-4-[(6,7-dimethoxy-4-quinolyl)oxy]-phenyl}-N′-(5-methyl-3-isoxazolyl) urea or hydrates of a hydrochloride salt. In certain embodiments, tivozanib is N-{2-chloro-4-[(6,7-dimethoxy-4-quinolyl)oxy]-phenyl}-N′-(5-methyl-3-isoxazolyl) urea monohydrochloric acid salt monohydrate. In certain embodiments, tivozanib is tivozanib hydrochloride having the chemical name 1-{2-chloro-4-[(6,7-dimethoxyquinolin-4-yl)oxy]phenyl}-3-(5-methylisoxazol-3-yl) urea hydrochloride hydrate, having the chemical structure

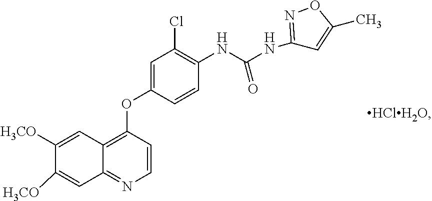 [SMILES: COc1cc2nccc(Oc3ccc(NC(=O)Nc4cc(C)on4)c(Cl)c3)c2cc1OC]

the molecular formula C₂₂H₁₉ClN₄O₅·HCl·H₂O, and a molecular weight of 509.34. Tivozanib hydrochloride is a white to light brown crystalline powder that is practically insoluble in water.

Treating (a cancer): As used herein, the terms “treating” or “treat” or “treatment” in the context of cancer, i.e., bile duct cancer including cholangiocarcinoma, refer to applying techniques, actions or therapies to a subject that (a) slow tumor growth, (b) halt tumor growth, (c) promote tumor regression or disappearance, (d) ameliorate a symptom of the cancer, (e) cure the cancer, or (f) prolong survival of the subject, or applying techniques, actions or therapies to a subject in an attempt to achieve any of (a)-(f) regardless of whether the individual actually responds to the technique, action or therapy.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, or weight of the tumor. A tumor that invades the surrounding tissue and/or can metastasize to a location distant from the original/primary tumor is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

### II. Methods of Treatment

The disclosure involves methods of treating bile duct cancers, for example, cholangiocarcinoma with tivozanib.

According to certain embodiments of the disclosure, treatment with tivozanib is indicated as long as a clinical benefit is observed in the subject or until unacceptable toxicity occurs.

According to one embodiment, the disclosure provides methods of treating bile duct cancer, such as cholangiocarcinoma in a subject in need thereof. The method includes administering an effective amount of tivozanib to the subject, thereby to treat the bile duct cancer.

According to one embodiment, the disclosure provides methods of treating bile duct cancer, such as cholangiocarcinoma, in a subject in need thereof. The method includes administering an effective amount of tivozanib to a subject identified as having bile duct cancer that expresses exportin 7 (XPO7) and/or STE-20 like kinase (SLK), thereby to treat the bile duct cancer.

According to one embodiment, the disclosure provides methods of treating bile duct cancer, such as cholangiocarcinoma, in a subject in need thereof. The method includes administering an effective amount of tivozanib to a subject identified as having bile duct cancer that expresses exportin 7 (XPO7) and STE-20 like kinase (SLK), thereby to treat the bile duct cancer.

According to one embodiment, the disclosure provides methods of treating bile duct cancer, such as cholangiocarcinoma, in a subject in need thereof. The method includes administering an effective amount of tivozanib to a subject identified as having bile duct cancer that expresses exportin 7 (XPO7), thereby to treat the bile duct cancer.

According to one embodiment, the disclosure provides methods of treating bile duct cancer, such as cholangiocarcinoma, in a subject in need thereof. The method includes administering an effective amount of tivozanib to a subject identified as having bile duct cancer that expresses SLK, thereby to treat the bile duct cancer.

According to one embodiment, the disclosure provides methods of treating bile duct cancer, such as cholangiocarcinoma, in a subject in need thereof. The method includes administering an effective amount of tivozanib to a subject identified as having bile duct cancer that expresses exportin 7 (XPO7) in the cytoplasm of tumor cells of the bile duct cancer, thereby to treat the bile duct cancer.

According to one embodiment, the disclosure provides methods of treating bile duct cancer, such as cholangiocarcinoma, in a subject in need thereof. The method includes administering an effective amount of tivozanib to a subject identified as having bile duct cancer that expresses exportin 7 (XPO7) in the cytoplasm and nuclei of tumor cells of the bile duct cancer, thereby to treat the bile duct cancer.

According to one embodiment, the disclosure provides methods of treating bile duct cancer, such as cholangiocarcinoma, in a subject in need thereof. The method includes administering an effective amount of tivozanib to a subject identified as having bile duct cancer that expresses SLK in the cytoplasm of tumor cells of the bile duct cancer, thereby to treat the bile duct cancer.

According to one embodiment, the disclosure provides methods of treating bile duct cancer, such as cholangiocarcinoma, in a subject in need thereof. The method includes administering an effective amount of tivozanib to a subject identified as having bile duct cancer that expresses SLK in the cytoplasm and nuclei of tumor cells of the bile duct cancer, thereby to treat the bile duct cancer.

According to one embodiment, the disclosure provides methods of treating bile duct cancer, such as cholangiocarcinoma, in a subject in need thereof. The method includes administering an effective amount of tivozanib to a subject identified as having bile duct cancer that expresses exportin 7 (XPO7) in the cytoplasm of tumor cells of the bile duct cancer, thereby to treat the bile duct cancer. However, if a subject is identified as having XPO7 expression only in the nuclei of tumor cells of the bile duct cancer and not in the cytoplasm, the subject is not administered tivozanib.

According to one embodiment, the disclosure provides methods of treating bile duct cancer, such as cholangiocarcinoma, in a subject in need thereof. The method includes administering an effective amount of tivozanib to a subject identified as having bile duct cancer that expresses SLK in the cytoplasm of tumor cells of the bile duct cancer, thereby to treat the bile duct cancer. However, if a subject is identified as having SLK expression only in the nuclei of tumor cells of the bile duct cancer and not in the cytoplasm, the subject is not administered tivozanib.

According to another embodiment, the disclosure provides methods of identifying a subject having a bile duct cancer, such as cholangiocarcinoma, who is eligible for treatment with tivozanib. The method includes determining whether the bile duct cancer expresses XPO7 and SLK. A subject is identified as being eligible for treatment with tivozanib if the bile duct cancer expresses XPO7 and SLK.

According to another embodiment, the disclosure provides methods of identifying a subject having a bile duct cancer, such as cholangiocarcinoma, who is eligible for treatment with tivozanib. The method includes determining whether the bile duct cancer expresses XPO7 and SLK. A subject is identified as being eligible for treatment with tivozanib if the bile duct cancer expresses XPO7 and SLK in the cytoplasm of bile duct tumor cells from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses XPO7 and SLK. A subject is identified as being eligible for treatment with tivozanib if the bile duct cancer expresses XPO7 and SLK in the cytoplasm and nuclei of bile duct tumor cells from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses XPO7 and SLK. A subject is identified as being eligible for treatment with tivozanib if the bile duct cancer expresses XPO7 in the cytoplasm of bile duct tumor cells from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses XPO7 and SLK. A subject is identified as being eligible for treatment with tivozanib if the bile duct cancer expresses XPO7 in the cytoplasm and nuclei of bile duct tumor cells from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses XPO7 and SLK. A subject is identified as being eligible for treatment with tivozanib if the bile duct cancer expresses SLK in the cytoplasm of bile duct tumor cells from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses XPO7 and SLK. A subject is identified as being eligible for treatment with tivozanib if the bile duct cancer expresses SLK in the cytoplasm and nuclei of bile duct tumor cells from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses XPO7 or SLK. A subject is identified as being ineligible for treatment with tivozanib if the bile duct cancer does not express XPO7 or SLK.

According to another embodiment, the disclosure provides methods of identifying a subject having a bile duct cancer, such as cholangiocarcinoma, who is eligible for treatment with tivozanib. The method includes determining whether the bile duct cancer expresses XPO7 and SLK. A subject is identified as being ineligible for treatment with tivozanib if the bile duct cancer does not express XPO7 and SLK.

According to another embodiment, the disclosure provides methods of identifying a subject having a bile duct cancer, such as cholangiocarcinoma, who is eligible for treatment with tivozanib. The method includes determining whether the bile duct cancer expresses XPO7. A subject is identified as being eligible for treatment with tivozanib if the bile duct cancer expresses XPO7.

According to another embodiment, the disclosure provides methods of identifying a subject having a bile duct cancer, such as cholangiocarcinoma, who is eligible for treatment with tivozanib. The method includes determining whether the bile duct cancer expresses XPO7. A subject is identified as being eligible for treatment with tivozanib if the bile duct cancer expresses XPO7.

According to another embodiment, the disclosure provides methods of identifying a subject having a bile duct cancer, such as cholangiocarcinoma, who is eligible for treatment with tivozanib. The method includes determining whether the bile duct cancer expresses XPO7. A subject is identified as being eligible for treatment with tivozanib if the bile duct cancer expresses XPO7 in the cytoplasm of bile duct tumor cells from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses XPO7. A subject is identified as being eligible for treatment with tivozanib if the bile duct cancer expresses XPO7 in the cytoplasm and nuclei of bile duct tumor cells from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses XPO7. A subject is identified as being ineligible for treatment with tivozanib if the bile duct cancer expresses XPO7 in the nuclei of bile duct tumor cells, but not in the cytoplasm from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses XPO7. A subject is identified as being ineligible for treatment with tivozanib if the bile duct cancer does not express XPO7.

According to another embodiment, the disclosure provides methods of identifying a subject having a bile duct cancer, such as cholangiocarcinoma, who is eligible for treatment with tivozanib. The method includes determining whether the bile duct cancer expresses SLK. A subject is identified as being ineligible for treatment with tivozanib if the bile duct cancer expresses SLK in the nuclei of bile duct tumor cells, but not in the cytoplasm from the bile duct cancer.

According to another embodiment, the disclosure provides methods of identifying a subject having a bile duct cancer, such as cholangiocarcinoma, who is eligible for treatment with tivozanib. The method includes determining whether the bile duct cancer expresses SLK. A subject is identified as being ineligible for treatment with tivozanib if the bile duct cancer does not express SLK.

### III. Detecting Expression of SLK and XPO7

Known methods can be used to detect expression of SLK or XPO7 in bile duct cancers, such as cholangiocarcinoma. For example, a tissue sample may be taken from the bile duct cancer and subjected to an immunoassay. In one example, a tissue sample from a bile duct cancer is from a formalin-fixed paraffin-embedded (FFPE) block. In one example, a tissue sample from a bile duct cancer is a fresh tumor biopsy, such as a core biopsy. In one example, a tissue sample from a bile duct cancer is a fine needle aspirate. In one example, immunohistochemical testing is performed with an anti-XPO7 or anti-SLK antibody. For example, the anti-XPO7 or anti-SLK antibody may be fluorescently labeled to permit identification of XPO7 or SLK by fluorescent immunohistochemical evaluation, the anti-XPO7 or anti-SLK antibody may be labeled with a metal, such as gold nanoparticles to permit identification of XPO7 or SLK by electron microscopy, or the anti-XPO7 or anti-SLK antibody may be used with a secondary antibody conjugated to a fluorescent dye or other identifiable label (such as a gold nanoparticle, enzyme, and the like). For example, XPO7 antibodies can be made against the C-terminus of XPO7 as provided by Mingot et al., EMBO J. 2004 Aug. 18; 23 (16): 3227-36 or against different isoforms of XPO7, such as exon 1a and 1b isoforms. XPO7 polyclonal or monoclonal antibodies can be generated according to methods known in the art. Suitable XPO? antibodies are also commercially available, for example, from Proteintech (Rosemont, IL) or Novus Biological (Centennial, CO), and can be used according to standard protocols to detect expression of XPO7 in bile duct cancer tissue samples, for example, with secondary fluorescently conjugated antibodies. Tissue samples can be imaged by employing light, electron, or fluorescence microscopy to view signal, such as a fluorescent signal. High throughput detection systems can also be employed. Appropriate counter-stains can be used to highlight emphasize the detection of XPO7, SLK, or both according to standard immunohistochemistry methods. The Examples below provide exemplary methods by which XPO7 expression may be detected.

Samples can be immunostained with an anti-XPO7 antibody, anti-SLK antibody, or both, which may include a label, such as a fluorophore. If needed, the sample can subsequently incubated with a labeled secondary antibody. Appropriate positive and/or negative controls can be included. After staining, the samples are analyzed for the presence of XPO7, SLK, or both in the cytoplasm. Stained samples can be analyzed and scored by an expert pathologist blinded to clinical information, scored by an automated computer system, or both. The cytoplasmic staining pattern can be scored positive for weak intensity if at least 20% of the cells of the tumor had detectable cytoplasmic staining of XPO7, SLK, or both, or for any moderate or strong cytoplasmic staining of XPO7, SLK, or both. Thus, in some examples, a bile duct cancer that expresses cytoplasmic XPO7 (and is one that can be treated with tivozanib) is one wherein at least 20% of the cells have detectable XPO7 staining, such as at least 30%, at least 40%, at least 50%, at least 60%, at least 70%, at least 80%, at least 90%, at least 95%, at least 99%, or 100% of the cells have detectable XPO7 staining (for example at least this % of cells have detectable XPO7 protein in the cytoplasm). Thus, in some examples, a bile duct cancer that expresses cytoplasmic SLK (and is one that can be treated with tivozanib) is one wherein at least 20% of the cells have detectable SLK staining, such as at least 30%, at least 40%, at least 50%, at least 60%, at least 70%, at least 80%, at least 90%, at least 95%, at least 99%, or 100% of the cells have detectable SLK staining (for example at least this % of cells have detectable SLK protein in the cytoplasm). In some examples, combinations of these are achieved. In one example, an H-score is assigned using the following formula: [1×(% cells 1+)+2×(% cells 2+)+3×(% cells 3+)]. The final score, ranging from 0 to 300, gives more relative weight to higher-intensity membrane staining in a given tumor sample. The sample can then be considered positive or negative on the basis of a specific discriminatory threshold (such as greater than 50, greater than 100, or greater than 150). In one example, a cancer is considered positive for cytoplasmic expression of XPO7 if the sample has an H-score of 51 or greater, 101 or greater, 126 or greater, 150 or greater, or 200 or greater. In one example, a cancer is considered positive for cytoplasmic expression of SLK if the sample has an H-score of 51 or greater, 101 or greater, 126 or greater, 150 or greater, or 200 or greater. In one example staining intensity is scored as weak (1), moderate (2), or strong (3).

In some examples, immunoprecipitation is used to detect the presence and localization pattern (e.g., nuclear or cytoplasmic) of XPO7 and/or SLK in a tissue sample, such as a bile duct cancer sample. Tissue samples are fractionated into nuclear and cytoplasmic fractions, for example using a commercially available kit. The proteins in each fraction can be extracted. In some examples the fractions are pre-cleared with agarose beads to reduce the presence of other proteins. The nuclear and cytoplasmic fractions can be incubated with an anti-XPO7 antibody, anti-SLK antibody, or both, which may include a solid substrate, such as agarose or magnetic beads. If needed, the sample can subsequently incubated with a secondary antibody containing a solid substrate. Appropriate positive and/or negative controls can be included. After allowing the antibodies appropriate time to bind to their target, samples are washed and the antibody-protein-solid support conjugates retrieved, for example by centrifugation or binding to a magnetic column. In some examples, the bound proteins are eluted from the solid support. The proteins present can then be detected by immunoblotting, such as Western blotting. The amount of XPO7, SLK, or both, present in the cytoplasmic and nuclear fractions can then be assessed, for example qualitatively or quantitatively. In one example, the bile duct cancer is determined to be one that expresses XPO7, SLK, or both, cytoplasmically if there is at least 20% more detectable XPO7, SLK, or both, in the cytoplasm fraction as compared to the nuclear fraction. Thus, in some examples, a bile duct cancer that expresses cytoplasmic XPO7 (and is one that can be treated with tivozanib) is one wherein there is at least 20% more detectable XPO7 in the cytoplasm fraction as compared to the nuclear fraction, such as at least 30%, at least 40%, at least 50%, at least 60%, at least 70%, at least 80%, at least 90%, at least 95%, at least 99%, at least 100%, at least 200%, at least 300%, at least 400%, or at least 500% more detectable XPO7 in the cytoplasm fraction as compared to the nuclear fraction. Thus, in some examples, a bile duct cancer that expresses cytoplasmic SLK (and is one that can be treated with tivozanib) is one wherein there is at least 20% more detectable SLK in the cytoplasm fraction as compared to the nuclear fraction, such as at least 30%, at least 40%, at least 50%, at least 60%, at least 70%, at least 80%, at least 90%, at least 95%, at least 99%, at least 100%, at least 200%, at least 300%, at least 400%, or at least 500% more detectable SLK in the cytoplasm fraction as compared to the nuclear fraction. In some examples, combinations of these are achieved.

In some embodiments, XPO7 or SLK expression is detected in the cytoplasm of cells from a bile duct cancer tumor sample. In some embodiments, XPO7 or SLK expression is detected in the nuclei of cells from a bile duct cancer tumor sample. In some embodiments, XPO7 or SLK expression is detected in the nuclei but not in the cytoplasm of cells from a bile duct cancer tumor sample. In some embodiments, XPO7 or SLK expression is not detected in the cytoplasm or nuclei of cells from a bile duct cancer tumor sample.

### IV. Exemplary Subjects

In some embodiments, a subject is treated according to methods of the invention if the subject has bile duct cancer, such as cholangiocarcinoma, and the subject's bile duct cancer has not been previously treated with chemotherapy or immunotherapy (such as a checkpoint inhibitor, e.g., PD-1, PD-L1, or CTLA-4 inhibitors). In some embodiments, a subject is treated according to methods of the disclosure if the subject has bile duct cancer, such as cholangiocarcinoma, and the subject's bile duct cancer has not been previously treated with chemotherapy, immunotherapy (such as a checkpoint inhibitor, e.g., PD-1, PD-L1, or CTLA-4 inhibitors), or other non-surgical intervention such as radiation. In some embodiments, a subject is treated according to methods of the disclosure if the subject has bile duct cancer, such as cholangiocarcinoma, and the subject's bile duct cancer has not been previously treated with chemotherapy, immunotherapy (such as a checkpoint inhibitor, e.g., PD-1, PD-L1, or CTLA-4 inhibitors), but has been treated with radiation and/or the cancer has been surgically resected. In some embodiments, a subject having bile duct cancer, such as cholangiocarcinoma, is treated with tivozanib as first line therapy. In some embodiments, a subject having advanced or metastatic bile duct cancer, such as cholangiocarcinoma, is treated with tivozanib as first line therapy. In some embodiments, a subject having advanced or metastatic bile duct cancer, such as cholangiocarcinoma, is treated with tivozanib as first line therapy after radiation and surgical resection, where the bile duct cancer recurs. In some embodiments, a subject having bile duct cancer, such as cholangiocarcinoma, is treated with tivozanib as first line therapy after radiation and/or surgical resection of a bile duct tumor, such as cholangiocarcinoma and the subject has not previously received chemotherapy or immunotherapy. The bile duct cancer may be advanced or metastatic or recurrent.

In some embodiments, the subject with bile duct cancer, such as cholangiocarcinoma, is treated with tivozanib where the bile duct cancer was previously treated with chemotherapy. For example, the tivozanib is a second line therapy for bile duct cancer, such as cholangiocarcinoma. For example, the subject's bile duct cancer has been treated with at least one chemotherapy.

Chemotherapies that the subject may have received include antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent.

In some embodiments, the chemotherapy is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

In some embodiments, the chemotherapy is an antimetabolite. The antimetabolite can be, for example, a purine antagonist (e.g., azothioprine or mycophenolate mofetil), a dihydrofolate reductase inhibitor (e.g., methotrexate), acyclovir, ganciclovir, zidovudine, vidarabine, ribavarin, azidothymidine, cytidine arabinoside, amantadine, dideoxyuridine, iododeoxyuridine, poscarnet, or trifluridine.

In some embodiments, the subject's bile duct cancer was previously treated with a platinum chemotherapy such as cisplatin, oxaliplatin, or carboplatin. In some embodiments, the subject's bile duct cancer was previously treated with cisplatin. For example, the tivozanib is a second or later line therapy for bile duct cancer, such as cholangiocarcinoma.

In some embodiments, the subject's bile duct cancer was previously treated with an antimetabolite. In some embodiments, the antimetabolite was gemcitabine or capecitabine. In some embodiments, the subject's bile duct cancer was previously treated with gemcitabine. For example, the tivozanib is a second or later line therapy for bile duct cancer, such as cholangiocarcinoma.

In some embodiments, the subject's bile duct cancer, such as cholangiocarcinoma, was previously treated with gemcitabine and cisplatin. For example, the tivozanib is a second or later line therapy for bile duct cancer, such as cholangiocarcinoma. In some embodiments, the subject with bile duct cancer, such as cholangiocarcinoma, is treated with tivozanib where the bile duct cancer was previously treated with gemcitabine and cisplatin.

In some embodiments, the subject's bile duct cancer, such as cholangiocarcinoma, was previously treated with gemcitabine and cisplatin and 5-fluorouracil. For example, the tivozanib is a second line therapy for bile duct cancer, such as cholangiocarcinoma.

In some embodiments, the subject's bile duct cancer, such as cholangiocarcinoma, was previously treated with a fluoropyrimidine, for example 5-fluorouracil.

In some embodiments, the subject's bile duct cancer, such as cholangiocarcinoma, was previously treated with an FGFR2 inhibitor. For example, the FGFR2 inhibitor is pemigatinib or infigratinib.

In some embodiments, the subject's bile duct cancer, such as cholangiocarcinoma, was previously treated with an isocitrate dehydrogenase 1 (IDH1) inhibitor. For example, the IDH1 inhibitor is ivosidenib.

In some embodiments, the subject's bile duct cancer, such as cholangiocarcinoma, was previously treated with a checkpoint inhibitor. For example, the checkpoint inhibitor is an anti-PD1, anti-PD-L1, or CTLA-4 inhibitor. For example, the checkpoint inhibitor is pembrolizumab, nivolumab, cemiplumab, atezolizumab, avelumab, durvalumab, ipilimumab, tremelimumab or tisotumab. For example, the tivozanib is a second or later line therapy for bile duct cancer, such as cholangiocarcinoma.

Embodiments disclosed herein for treating bile duct cancer are also intended for treating advanced bile duct cancer, metastatic bile duct cancer, recurrent bile duct cancer, and surgically unresectable bile duct cancer. Embodiments disclosed herein for treating bile duct cancer are also intended for treating cholangiocarcinoma, including advanced cholangiocarcinoma, metastatic cholangiocarcinoma, recurrent cholangiocarcinoma, and surgically unresectable cholangiocarcinoma.

In one embodiment, the disclosure provides methods of treating bile duct cancer, such as cholangiocarcinoma, where the cancer is recurrent, metastatic, or advanced, in a subject, where an effective amount of tivozanib is administered to a subject identified as having bile duct cancer that express XPO7 and the cancer has previously been treated with one or more chemotherapies. For example, tivozanib is a second or later line chemotherapeutic treatment. For example, XPO7 is detected in the cytoplasm of cells of the bile duct cancer. For example, the cancer has previously been treated with gemcitabine and cisplatin.

In one embodiment, the disclosure provides methods of treating bile duct cancer, such as cholangiocarcinoma, where the cancer is recurrent, metastatic, or advanced, in a subject, where an effective amount of tivozanib is administered to a subject identified as having bile duct cancer that express SLK and the cancer has previously been treated with one or more chemotherapies. For example, tivozanib is a second or later line chemotherapeutic treatment. For example, SLK is detected in the cytoplasm of cells of the bile duct cancer. For example, the cancer has previously been treated with gemcitabine and cisplatin.

In one embodiment, the disclosure provides methods of treating bile duct cancer, such as cholangiocarcinoma, where the cancer is recurrent, metastatic, or advanced, in a subject, where an effective amount of tivozanib is administered to a subject identified as having bile duct cancer that express SLK and the cancer has previously been treated with one or more chemotherapies or immunotherapies. For example, tivozanib is a second or later line chemotherapeutic treatment. For example, SLK is detected in the cytoplasm of cells of the bile duct cancer.

In one embodiment, the disclosure provides methods of treating bile duct cancer, such as cholangiocarcinoma, where the cancer is recurrent, metastatic, or advanced, in a subject, where an effective amount of tivozanib is administered to a subject identified as having bile duct cancer that express XPO7 and the cancer has not been previously been treated with chemotherapy or immunotherapy. For example, tivozanib is a first line chemotherapeutic treatment. For example, XPO7 is detected in the cytoplasm of cells of the bile duct cancer. For example, the bile duct cancer has been previously treated only with radiation or by surgical resection prior to treatment with tivozanib.

In one embodiment, the disclosure provides methods of treating bile duct cancer, such as cholangiocarcinoma, where the cancer is recurrent, metastatic, or advanced, in a subject, where an effective amount of tivozanib is administered to a subject identified as having bile duct cancer that express SLK and the cancer has not been previously been treated with chemotherapy or immunotherapy. For example, tivozanib is a first line chemotherapeutic treatment. For example, XPO7 is detected in the cytoplasm of cells of the bile duct cancer. For example, the bile duct cancer has been previously treated only with radiation or by surgical resection prior to treatment with tivozanib.

The disclosure also provides a method of inhibiting SLK in a bile duct cancer. The method includes administering an effective amount of tivozanib to the bile duct cancer to inhibit SLK in the bile duct cancer. The bile duct cancer may be in in vitro, such as a tumor specimen, or other in vitro assay using tumor tissue or tumor cells. The bile duct cancer may be in a human patient. Tivozanib may be administered in dosages and according to administration methods as disclosed herein. Tivozanib may be administered to subjects having bile duct cancer to inhibit SLK in the cancer, according to the various types of subjects disclosed herein, e.g., subjects having received certain previous treatments for bile duct cancer, or subject having not received certain treatments.

### V. Dosage of Tivozanib

Exemplary effective amounts, dosages, or treatment regimens of tivozanib for treating bile duct cancer include 0.5-3 mg, 0.5-2 mg, 1-3 mg, 0.5-1.5 mg, 1.0-2.0 mg, 1.0-1.5 mg, 1.4-1.6 mg, 0.8-0.9 mg, 0.9-1.0 mg, 0.9-1.1 mg, 1.0-1.1 mg, 1.1-1.2 mg, 1.2-1.3 mg, 1.3-1.4 mg, 1.4-1.5 mg, 1.4-1.6 mg, 1.5-1.6 mg, 1.6-1.7 mg, 1.7-1.8 mg, 1.8-1.9 mg, 1.8-2.0 mg or 1.9-2.0 mg daily or every other day.

The amount administered will depend on variables such as the type and extent of disease or indication to be treated, the overall health of the patient, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study. Dosing frequency can vary, depending on factors such as route of administration, dosage amount, and the disease being treated. Exemplary dosing frequencies are once per day, once every other day, once every three days, once every four days, once every five days, once every six days, once per week and once every two weeks. In one embodiment, the dosing frequency is every day for 21 days with 7 days off. In another embodiment, the dosing frequency is every day for 28 days. In another embodiment, the dosing frequency is every other day for 28 days.

In one embodiment, the dosage of tivozanib for treating bile duct cancer is 1.5 mg daily. In another embodiment, the dosage is 1.0 mg daily. Additional exemplary effective amounts, dosages, or treatment regimens of tivozanib are described in U.S. Pat. Nos. 6,821,987, and 7,166,722 and in International Patent Application No. WO 2020/097106.

According to one embodiment, the dosage for treating bile duct cancer is 1.0 mg daily of tivozanib hydrochloride (equivalent to 0.89 mg tivozanib free base). According to another embodiment, the dosage is 1.5 mg daily of tivozanib hydrochloride (equivalent to 1.34 mg tivozanib free base). In one embodiment, the dose is 1.34 mg daily of tivozanib free base. In another embodiment, the dose is 0.89 mg daily of tivozanib free base.

According to one embodiment, a 1.5 mg daily dose of tivozanib for 21 days is reduced to 1.0 mg daily for 21 days when a subject experiences a ≥Grade 3 drug-related adverse event in the treatment of bile duct cancer, such as cholangiocarcinoma.

According to one embodiment, a 1.5 mg daily dose of tivozanib for 21 days is reduced to 1.0 mg every other day for 28 days when a subject experiences a ≥Grade 3 drug-related adverse event in the treatment of bile duct cancer, such as cholangiocarcinoma.

According to one embodiment, a 1.5 mg daily dose of tivozanib for 21 days is reduced to 1.5 mg every other day for 28 days or to 1.0 mg every day for 21 days for a subject experiencing moderate hepatic impairment (Child-Pugh class B or total bilirubin greater than 1.5 to 3 times ULN with any AST (aspartate transaminase)) in the treatment of bile duct cancer, such as cholangiocarcinoma.

According to one embodiment, a 1.5 mg daily dose of tivozanib is reduced to 1.0 mg every other day for a subject experiencing severe hepatic impairment (Child-Pugh class C or total bilirubin level greater than 3 to 10 times ULN with any AST) in the treatment of bile duct cancer, such as cholangiocarcinoma.

In one embodiment, the dose for treatment of bile duct cancer, such as cholangiocarcinoma, is 1.5 mg daily of tivozanib hydrochloride administered for 21 days followed by 7 days without administration, which constitutes a treatment cycle.

In one embodiment, the dose for treatment of bile duct cancer, such as cholangiocarcinoma, is 1.0 mg daily of tivozanib hydrochloride administered for 21 days followed by 7 days without administration, which constitutes a treatment cycle.

In one embodiment, the dose for treatment of bile duct cancer, such as cholangiocarcinoma, is 1.0 mg daily of tivozanib hydrochloride administered for 28 days, which constitutes a treatment cycle.

In one embodiment, the dose for treatment of bile duct cancer, such as cholangiocarcinoma, is 1.0 mg every other day of tivozanib hydrochloride administered for 28 days, which constitutes a treatment cycle.

In one embodiment, the dose for treatment of bile duct cancer, such as cholangiocarcinoma, is 1.5 mg every other day of tivozanib hydrochloride administered for 28 days, which constitutes a treatment cycle.

### VI. Administration Protocol

Tivozanib is administered to subjects having bile duct cancer, such as cholangiocarcinoma according to the methods of the disclosure. Tivozanib may be administered as an oral tablet or capsule or as an intravenous (IV) infusion. When administered as an oral tablet or capsule, the dosage of tivozanib may be provided in a single capsule or tablet or in two or more capsules or tablets. In one embodiment, tivozanib is administered as a single dose tablet. In one embodiment, for treatment of bile duct cancer, such as cholangiocarcinoma, tivozanib is administered as a tablet or capsule.

Exemplary effective amounts, dosages, or treatment regimens of tivozanib for the treatment of bile duct cancer, including cholangiocarcinoma, include administration on a repeating schedule of one dose (e.g., a single dosage contains 0.5-2.0 mg of tivozanib) per day for three weeks, followed by one week off (i.e., 3 weeks on, 1 week off). For example, tivozanib may be administered on a repeating schedule of 0.5-3 mg, 0.5-2 mg, 0.5-1.5 mg, 1.0-3.0 mg, 1.0-2.0 mg, 1.0-1.5 mg, or 1.4-1.6 mg per day for three weeks, followed by one week off (i.e., 3 weeks on, 1 week off). The period of time beginning from Day 1 of administration to the last day of the week off may be referred to as a treatment cycle. In other embodiments, tivozanib may be administered as one dose (e.g., a single dosage contains 0.5-2.0 mg of tivozanib) per day. For example, tivozanib may be administered at a dose of 0.5-3 mg, 0.5-2 mg, 0.5-1.5 mg, 1.0-3.0 mg, 1.0-2.0 mg, 1.0-1.5 mg or 1.4-1.6 mg, 1 mg, or 1.5 mg daily.

In one embodiment, tivozanib is administered in an amount of 1.5 mg per day to treat bile duct cancer, including cholangiocarcinoma. In another embodiment, tivozanib is administered in an amount of 1.0 mg per day. In another embodiment, tivozanib is administered in an amount of 1.5 mg per day every day for three weeks (i.e., 21 days), followed by one week (i.e., 7 days) with no dose of tivozanib (i.e., 3 weeks on, 1 week off), where three weeks on tivozanib and one week off constitutes a 4 week treatment cycle.

In one embodiment, tivozanib is orally administered in an amount of 1.5 mg daily for three weeks, followed by one week off, while in another embodiment, tivozanib is administered in an amount of 1.0 mg daily for three weeks, followed by one week without administration of tivozanib to treat bile duct cancer, including cholangiocarcinoma. According to these embodiments, three weeks on and one week off constitutes a 4 week treatment cycle.

In one embodiment, tivozanib is orally administered in an amount of 1.0 mg every other day for 4 weeks (i.e., 28 days), which constitutes a treatment cycle.

In one embodiment, tivozanib is orally administered in an amount of 1.5 mg daily and reduced to 1.0 mg daily when the subject experiences a ≥Grade 3 drug-related adverse event in the treatment of bile duct cancer, including cholangiocarcinoma. In this embodiment, the administration period for tivozanib is three weeks (starting from the first 1.5 mg dose), followed by one week without administration of tivozanib. According to this embodiment, three weeks on and one week off constitutes a 4-week treatment cycle.

In one embodiment, tivozanib is orally administered in an amount of 1.5 mg daily and is reduced to 1.5 mg every other day when the subject develops moderate hepatic impairment (Child-Pugh class B or total bilirubin greater than 1.5 to 3 times ULN with any AST) in the treatment of bile duct cancer, including cholangiocarcinoma In this embodiment, the administration period is 3 weeks (starting from the first 1.5 mg dose) followed by one week without administration. According to this embodiment, three weeks on and one week off constitutes a 4-week treatment cycle.

In one embodiment, tivozanib is orally administered in an amount of 1.5 mg daily and is reduced to 1.0 mg every other day when a subject develops severe hepatic impairment (Child-Pugh class C or total bilirubin level greater than 3 to 10 times ULN with any AST). In this embodiment, the administration period is 3 weeks followed by one week without administration. According to this embodiment, three weeks on and one week off constitutes a 4-week treatment cycle.

According to one embodiment, a subject undergoes one, two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, or 36 or more treatment cycles of tivozanib, for example, four-week treatment cycles of tivozanib, for example, where the treatment cycle is three weeks on, one week off. According to one embodiment, a treatment cycle (for example, a four-week treatment cycle) is repeated as long as the subject experiences a clinical benefit or until the subject experiences unacceptable toxicity.

In a further embodiment, tivozanib is administered as a capsule. In a further embodiment, the capsule contains gelatin. In yet another embodiment, the capsule contains gelatin and titanium dioxide.

In a further embodiment, tivozanib is formulated as a pharmaceutical composition with mannitol and magnesium stearate. In other embodiments, other pharmaceutically acceptable carriers may be used.

### EXAMPLES

The invention is further illustrated by the following examples. The following examples are provided for illustration purposes only, and are not to be construed as limiting the scope or content of the invention in any way.

### Example 1: Evaluation of XPO7 as a Biomarker of Tivozanib Response in Cholangiocarcinoma

To establish exportin 7 (XPO7) as a biomarker of aggressive disease in cholangiocarcinoma (CCA), proteomic characterization of tumor derived exosomes from about 20 patients with CCA and 20 patients with benign bile duct diagnoses were evaluated for expression of XPO7. Bile samples from 20 CCA patients and 20 patients with benign bile duct diagnoses were collected by intraoperative needle cannulation of the common bile duct and diluted and homogenized in 1 mL PBS. Exosomes were isolated by serial centrifugation at 500×g for 10 mins, 3000×g for 20 mins, 12000×g for 20 mins, and 100,000×g for 70 minutes. The pellet was resuspended in 2 mL PBS and further centrifuged at 100,000×g for 70 mins after which the pellet was resuspended in 100 μL and then the exosomes were characterized by transmission electron microscopy. The exosomes were then lysed with radiofrequency ablation and further characterized by mass spectrometry. Proteomic characterization of the exosomes revealed that XPO7 expression in CCA tumor derived exosomes was increased compared to normalized levels. Levels of XPO7 were 16.46 fold higher in CCA tumor exosomes as compared to normalized levels (p value of 0.002), as shown in Table 1 below. Other markers were also tested but XPO7 had the lowest p value, indicating it was statistically significantly correlated with CCA. For samples from patients with benign bile duct issues, XPO7 was not detectable.

TABLE 1
Proteomic Characterization of CCA Bile Exosomes

| Protein | Gene | Fold Change | P Value |
|---|---|---|---|
| Exportin-7 | XPO7 | 16.46 | 0.002 |
| Olfactiomedin-4 | OLFM4 | 6.95 | 0.01 |
| Integrin alpha-M | ITGAM | 3.73 | 0.04 |
| Myeloid cell nuclear | MNDA | 3.40 | 0.06 |
| differentiation | | | |
| antigen | | | |
| Fibronectin | FNI | 2.65 | 0.09 |

To determine expression of exportin 7 (XPO7) in cholangiocarcinoma tissue obtained from human patients, immunohistochemical staining was performed on benign bile duct tissue and bile duct tissues from approximately 80 cholangiocarcinoma patients using an anti-exportin 7 antibody. The expression of XPO7 protein was evaluated on both whole tissue sections and a non-overlapping tumor microarray (TMA). For the tissue sections, representative formalin-fixed paraffin-embedded (FFPE) tumor blocks were obtained for each patient and IHC studies were performed with a Leica Bond RX (Leica Biosystems) automated stainer on 5-μm tissue sections using the standard avidin-biotin peroxidase method. Sections were immunostained with a 1:200 dilution of rabbit anti-XPO7 antibody (NBP1-32350, Novus Biological). To ensure antibody specificity, rabbit immunoglobulins were used as negative controls. After staining, the sections were mounted with Permount for digital scanning with Pannoramic Confocal (3dHistech). Stained whole tissue slides were scored by an expert pathologist blinded to clinical information. The cytoplasmic staining pattern was scored positive for weak intensity if at least 20% of the tumor was positive, or for any moderate or strong staining. Staining intensity was scored as weak (1), moderate (2), or strong (3).

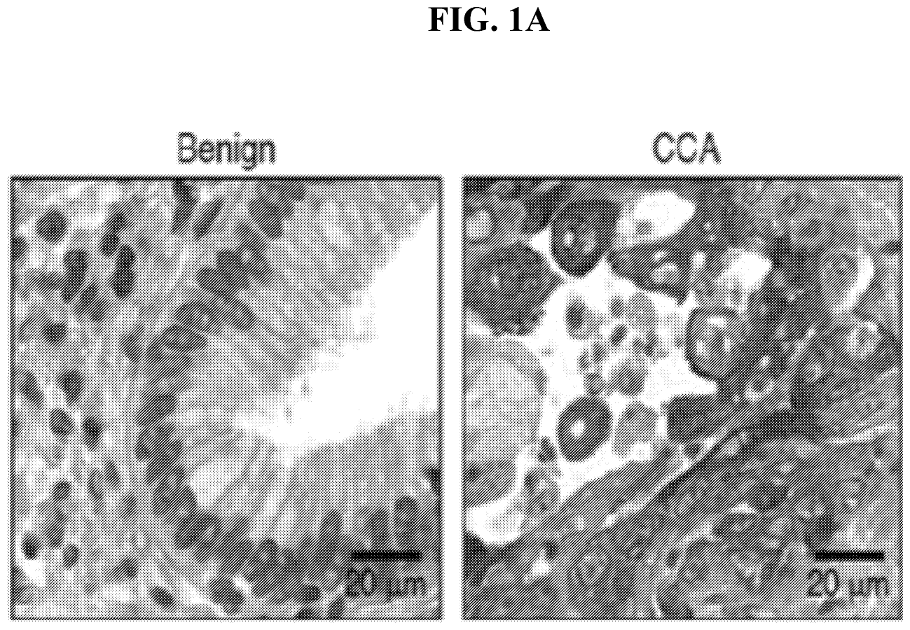

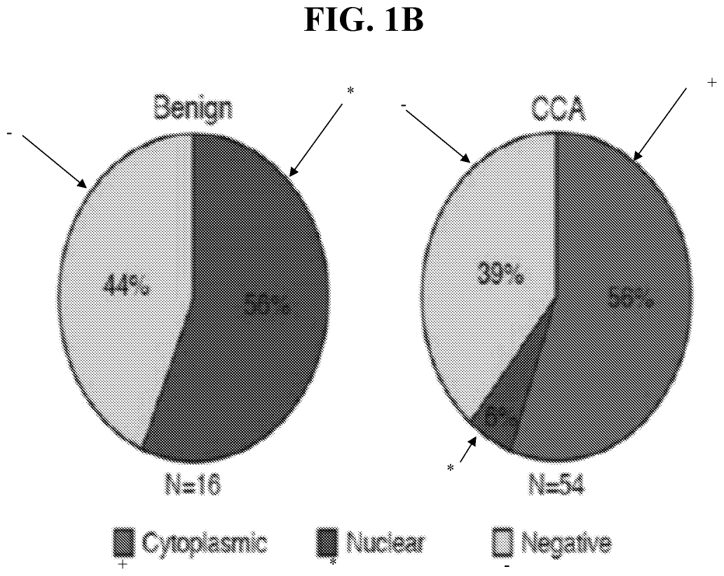

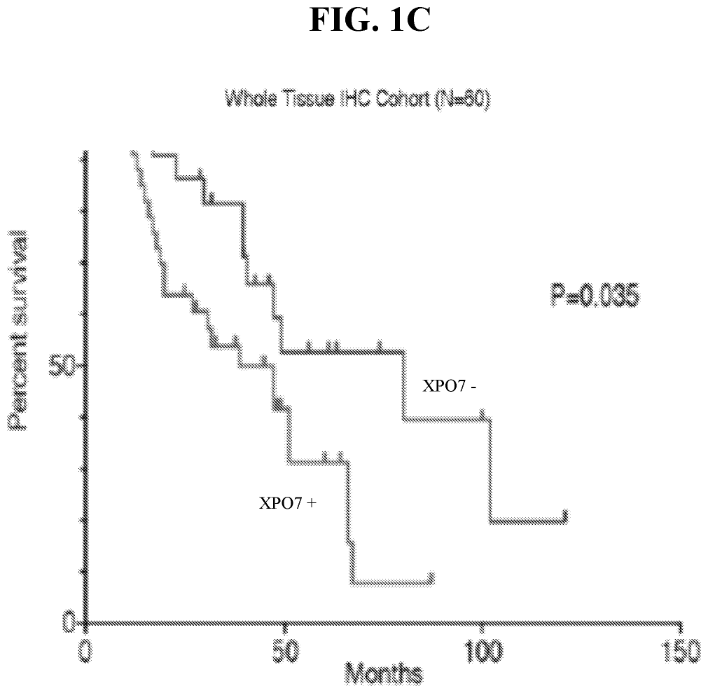

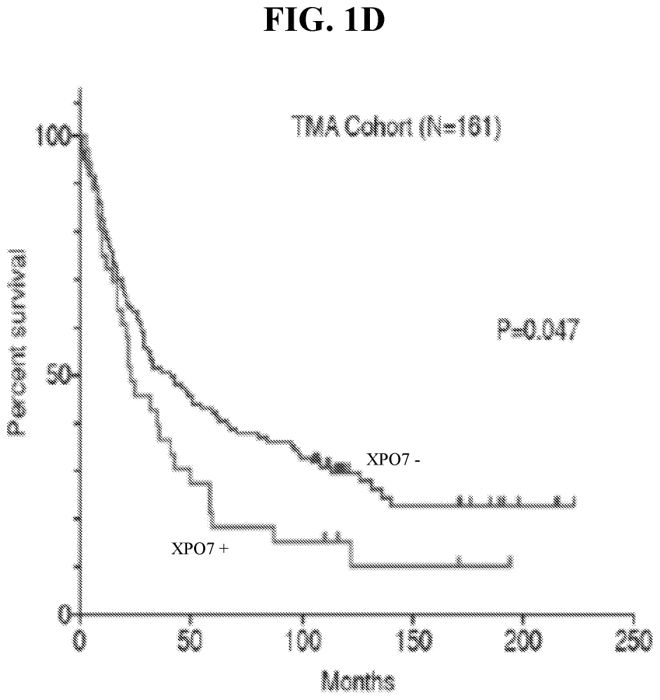

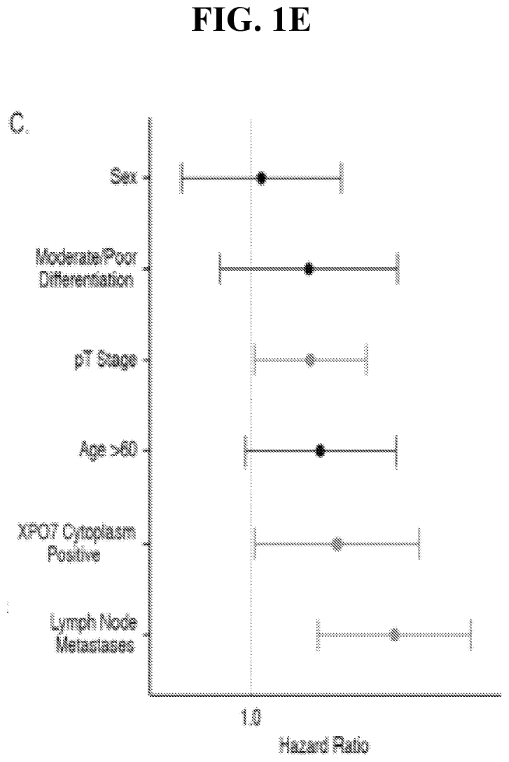

As shown in images of the immunohistochemical staining in FIG. 1A, both benign bile duct tissue and CCA tissue from cholangiocarcinoma patients exhibited expression of XPO7 expression in human cells. However, as the pie charts in FIG. 1B show, only CCA tissue was found to have increased cytoplasmic XPO7 expression, and then in only 56% of patient samples surveyed. However, for those patients, expression of XPO7 was associated with reduced survival in two independent cohorts. As shown in the Kaplan Meier survival curves in FIGS. 1C and 1D, the subjects with XPO7 expressing tumors in both the whole tissue cohort and the tumor microarray cohort had reduced chances of long-term survival compared to those without XPO7 (XPO7−)). Multivariate analysis performed and shown in FIG. 1E confirmed cytoplasmic XPO7 expression as a marker of poor prognosis in cholangiocarcinoma patients. The hazard ratio for XPO7 expression falls on the right side of the plot, showing a strong correlation with a poor outcome for these patients.

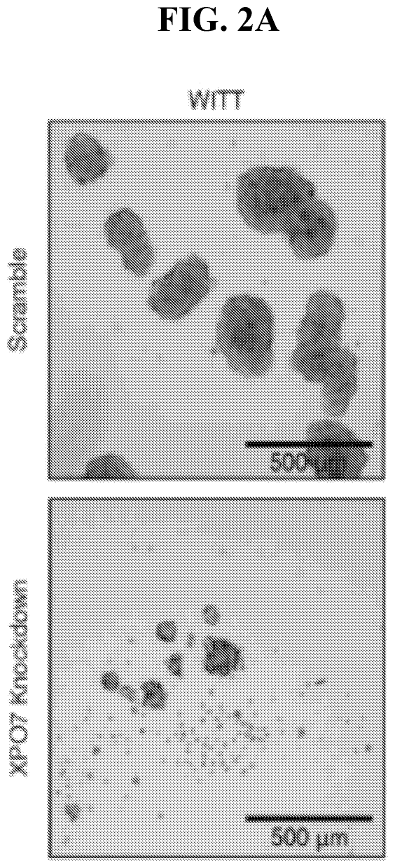

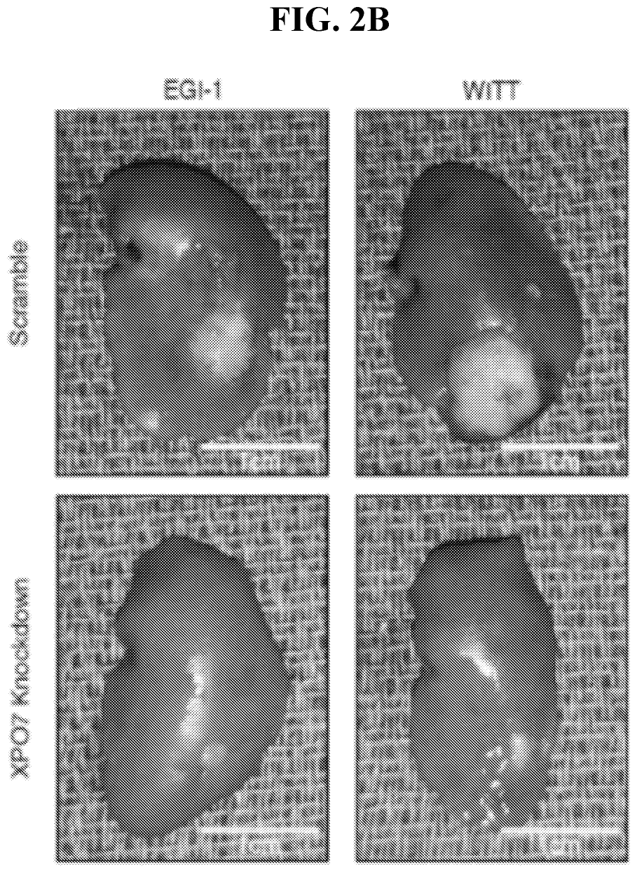

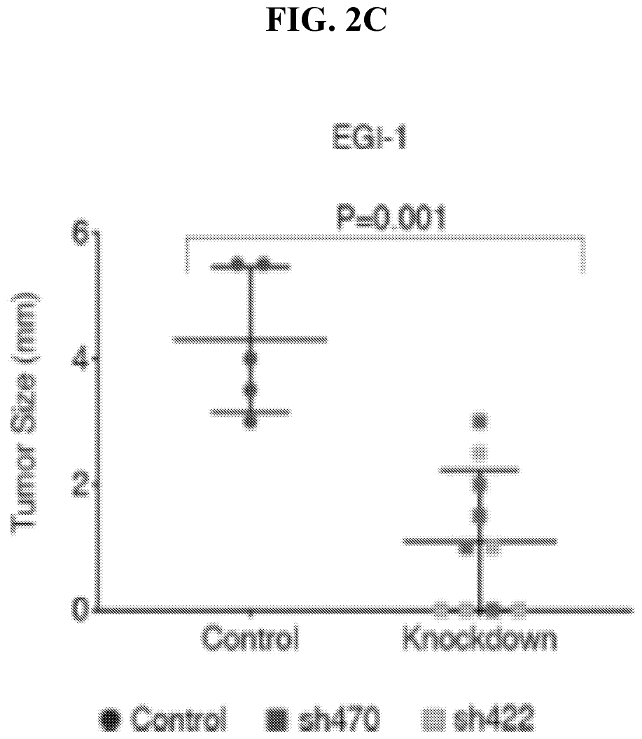

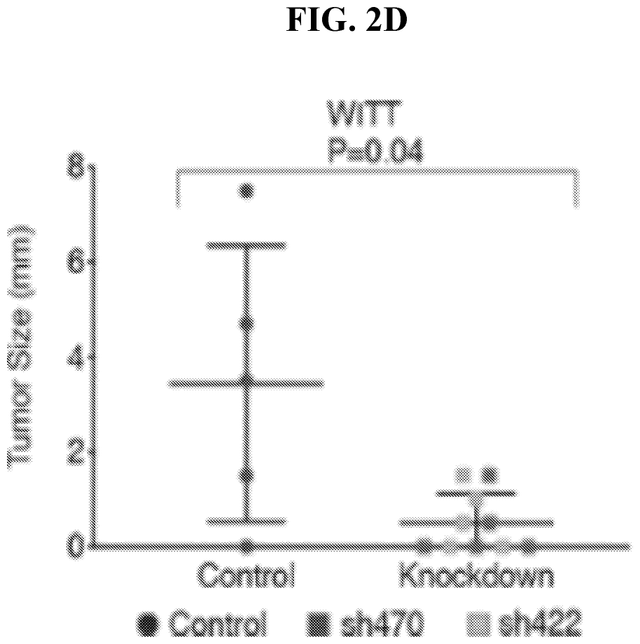

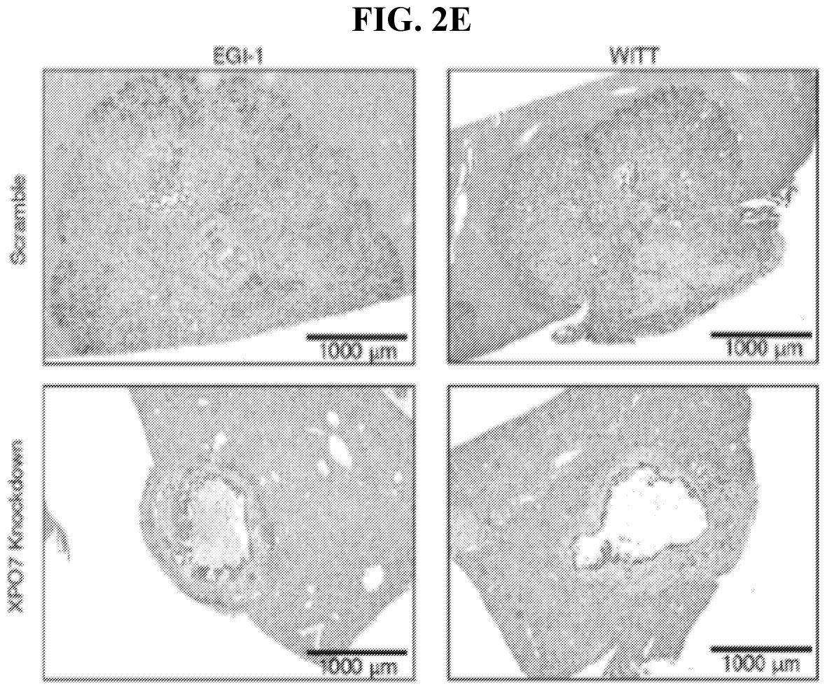

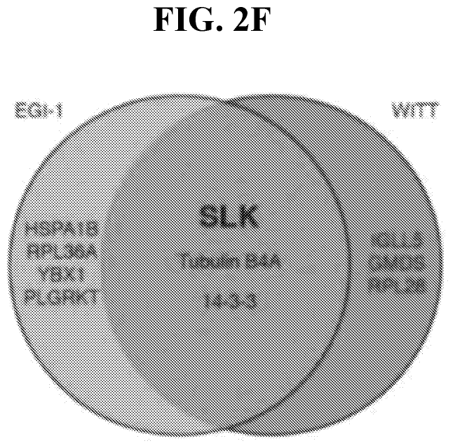

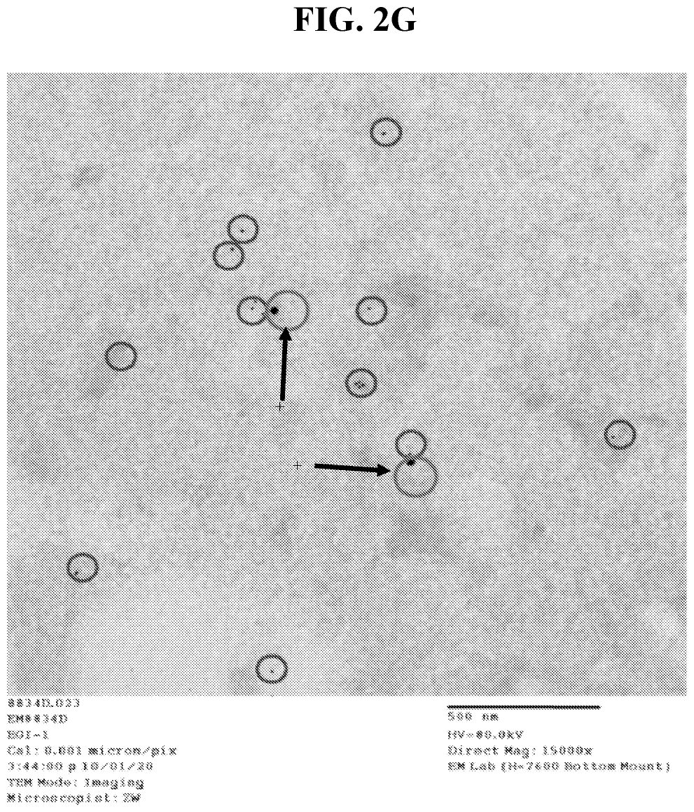

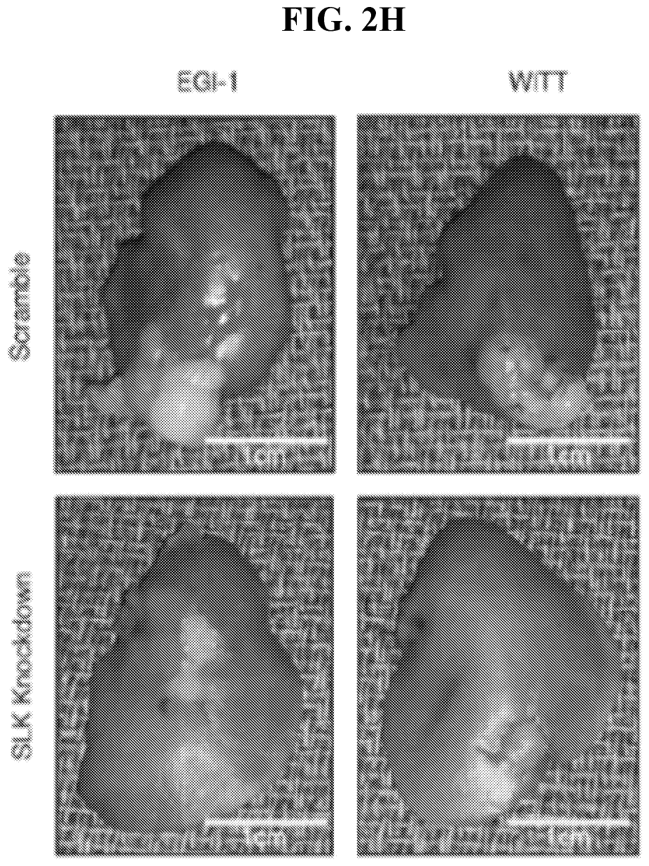

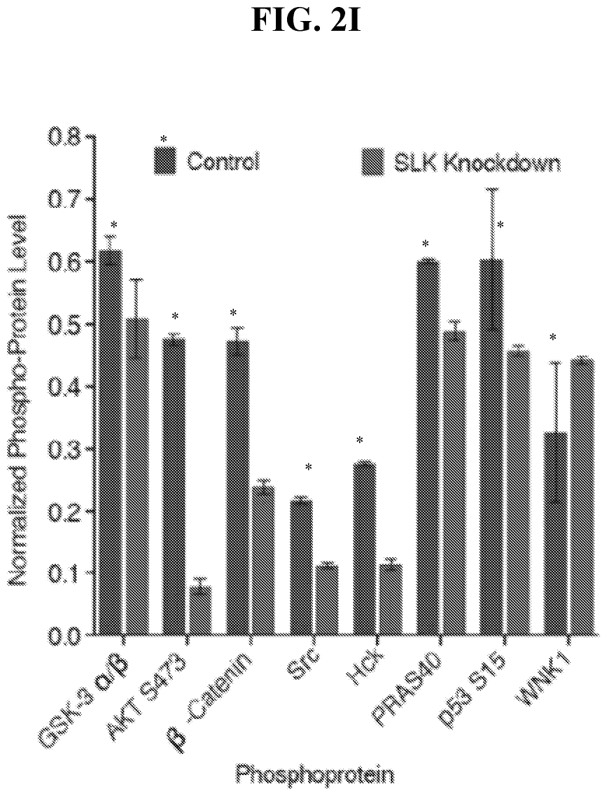

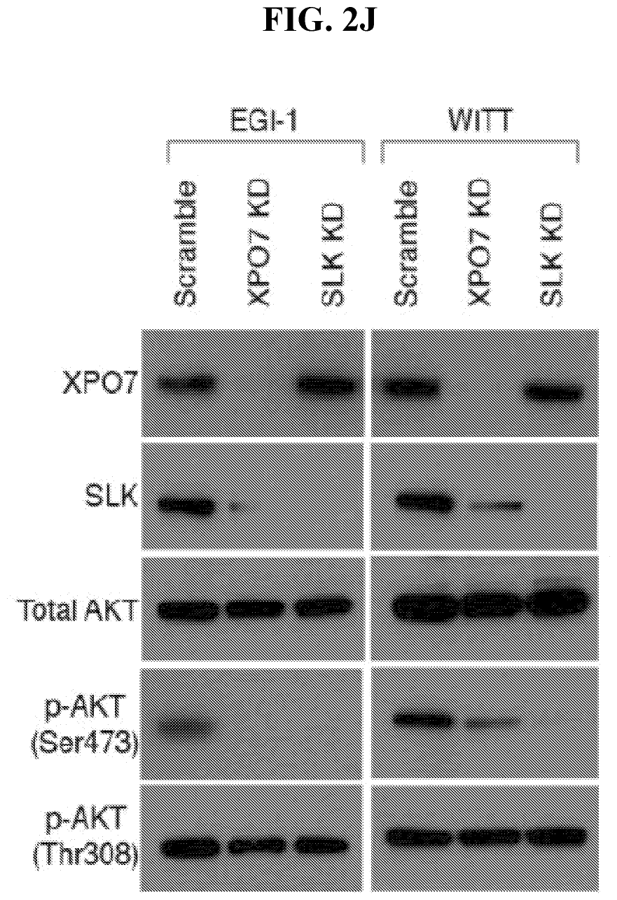

To assess the role of XPO7 in oncogenesis of cholangiocarcinoma, several XPO7 gene expression knock down experiments were performed in tumor organoids (tumorspheres) of cholangiocarcinoma cell lines in vitro and tumors in vivo. XPO7 knockdown resulted in a substantial and significant reduction of tumor organoid and tumor formation in vitro and in vivo, respectively (FIGS. 2A-2E). The CCA cell line WITT was used to show that XPO7 knockdown compared to the control resulted in significant reduction of tumor organoid formation (FIG. 2A). This was also shown in vivo in a xenograft tumor model using WITT and EGI-1 CCA cell line tumors. The photographs in FIG. 2B show that the tumors in the XPO7 knockdowns as compared to controls are smaller in size, a difference that was shown to be statistically significant based on the tumor size data in FIGS. 2C and 2D. The histological images of the EGI-1 and WITT tumors shown in FIG. 2E demonstrate that the XPO7 knockdown results in hypocellular and cystic-appearing tumors, as compared to the control.

### Example 2: Evaluation of the Interactions Between XPO7 and SLK in Cholangiocarcinoma

Immunoprecipitation-Mass Spectrometry was subsequently performed on the cytoplasm of cholangiocarcinoma cell lines to identify SLK as a binding partner of XPO7. Precipitated proteins were evaluated by mass spectrometry to identify proteins present in the cytoplasm. A Venn diagram provided in FIG. 2F shows the overlap between expression of potential binding partners in EGI-1 and WITT CCA cell lines, from which SLK was identified. Immuno-electron microscopy (EM) confirmed XPO7-SLK interaction in cytoplasm of cholangiocarcinoma cells. As showed in the micrograph in FIG. 2G, the small dots are XPO7, whereas the larger dots are SLK. At the position of the arrows, SLK is shown interacting with XPO7.

Similar to XPO7, SLK knockdown abrogated tumor growth in vivo in subcutaneous murine xenograft model. As shown in FIG. 2H, EGI-1 and WITT CCA tumors from a xenograft model subject to SLK knockdown have visibly smaller tumor masses than the EGI-1 and WITT CCA tumors subjected to the control (scramble). A phosphokinase array was used to assess the downstream molecular mechanism(s) following SLK knockdown. As shown in the bar graph in FIG. 2I, this assay showed a robust decrease in AKT Serine/Threonine Kinase (AKT) S473 phosphorylation upon SLK knockdown, implicating the role of this kinase in pro-tumorigenic signaling downstream of SLK. As shown in FIG. 2J, a Western blot confirmed that both XPO7 and SLK knockdown resulted in a decrease in AKT S473 phosphorylation compared to control (scramble).

### Example 3: Evaluation of Tivozanib as an Inhibitor of SLK and XPO7 in Cholangiocarcinoma

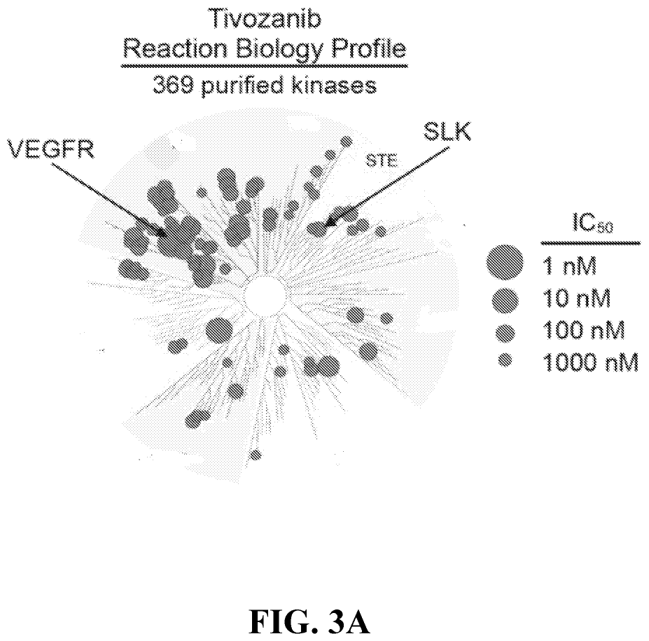

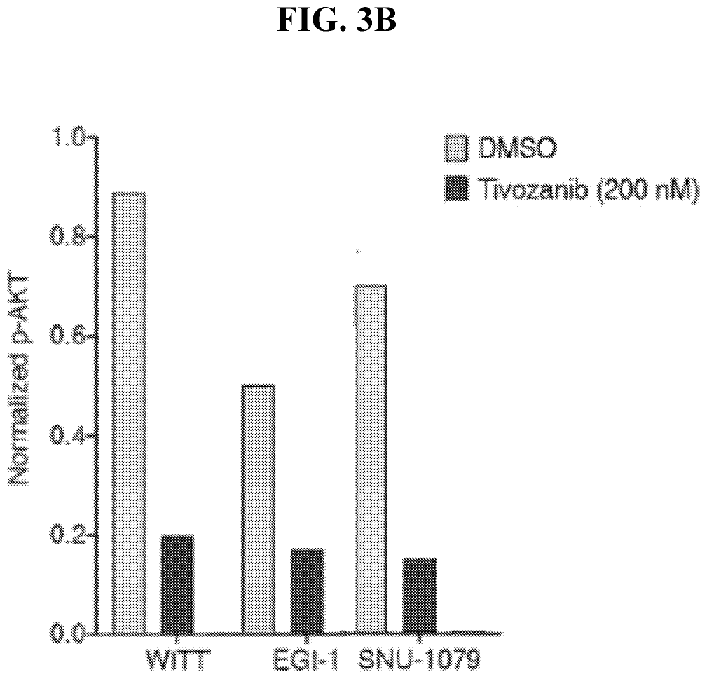

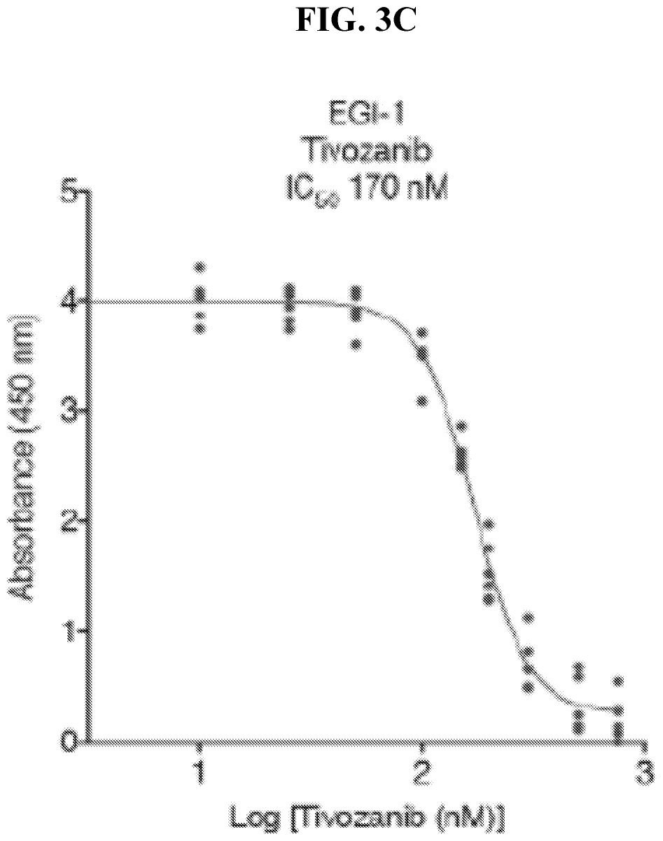

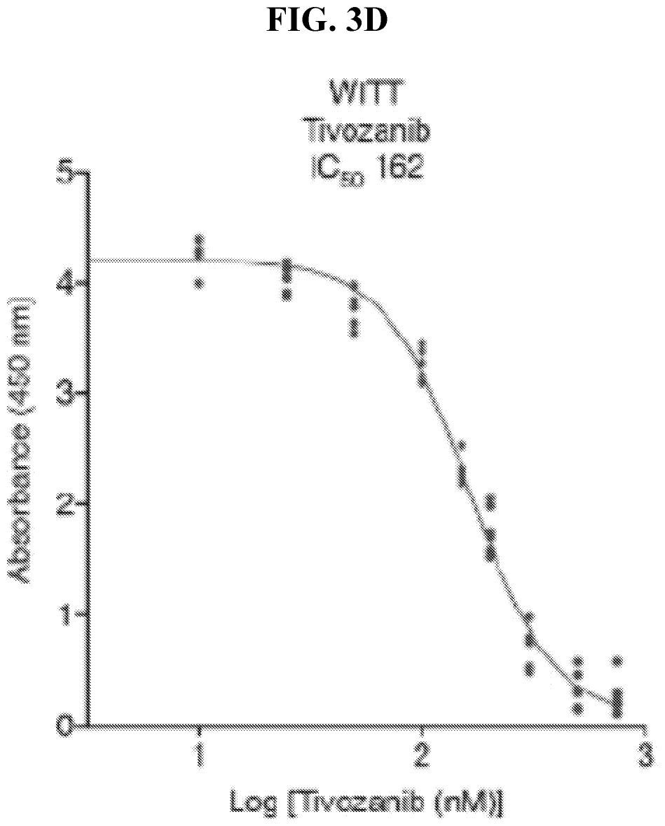

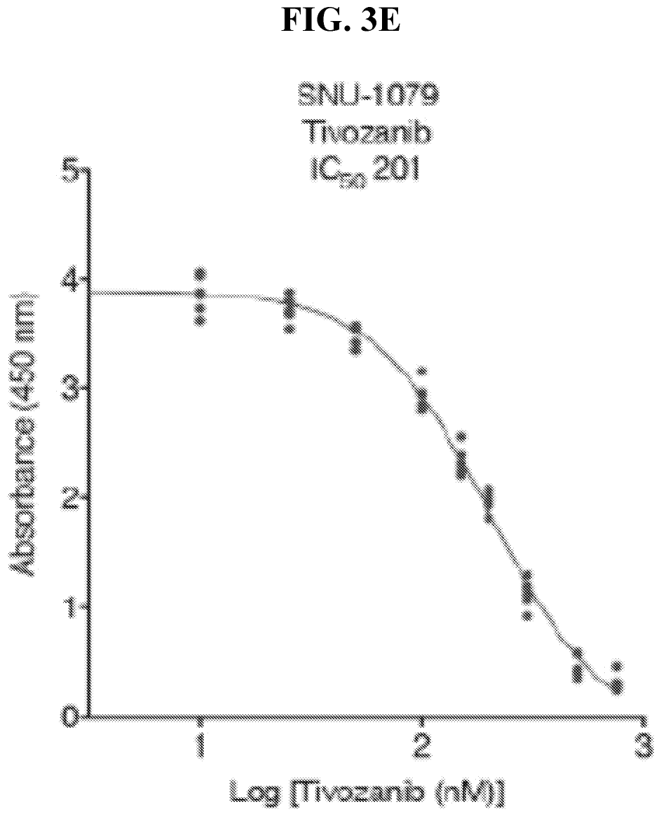

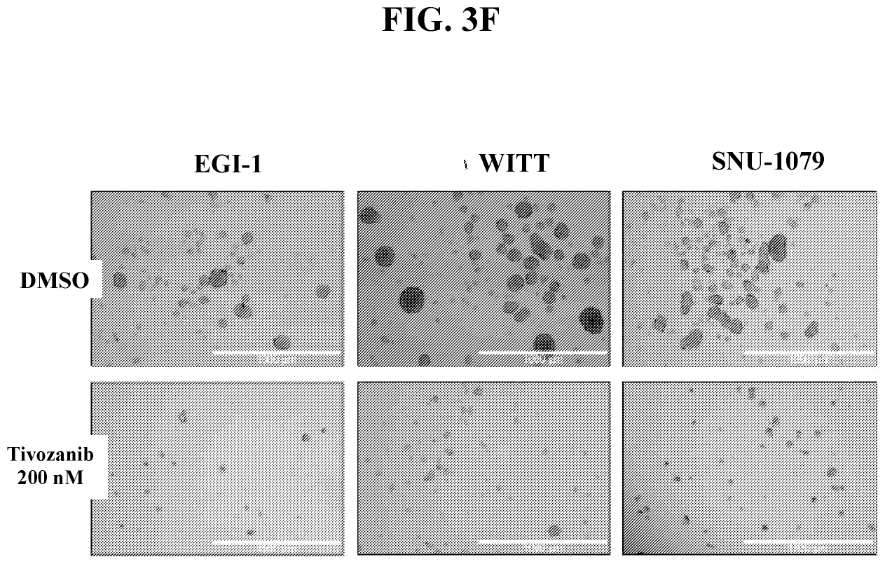

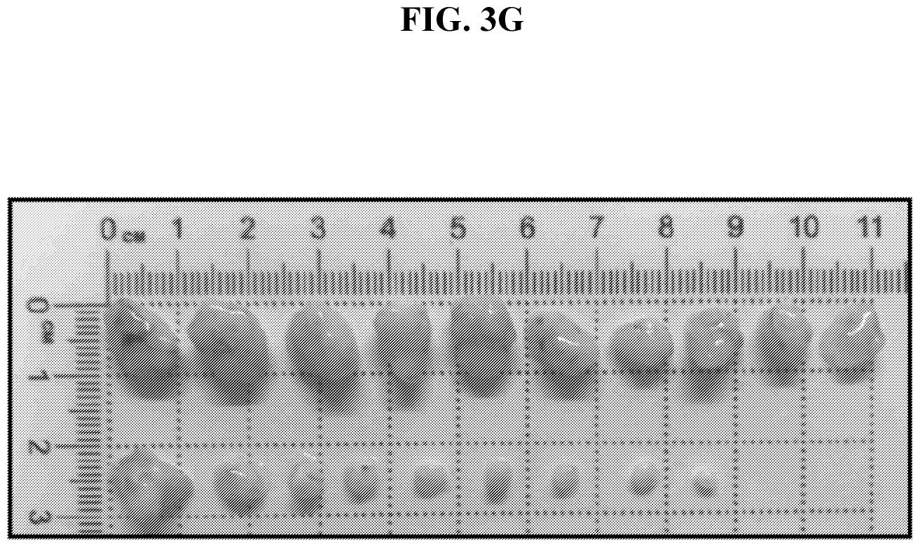

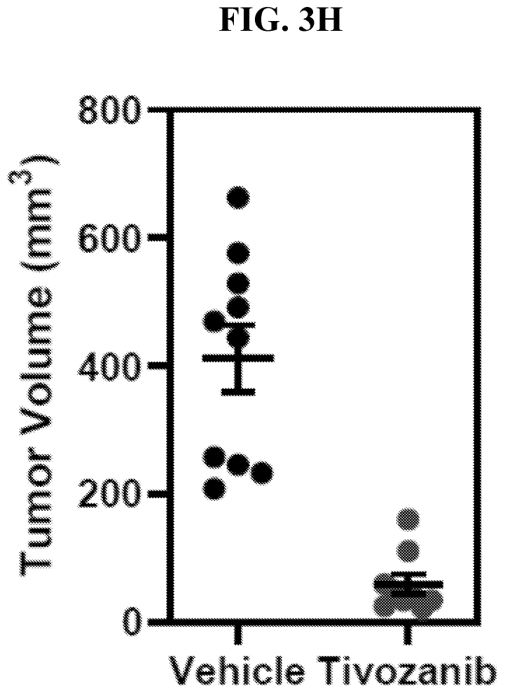

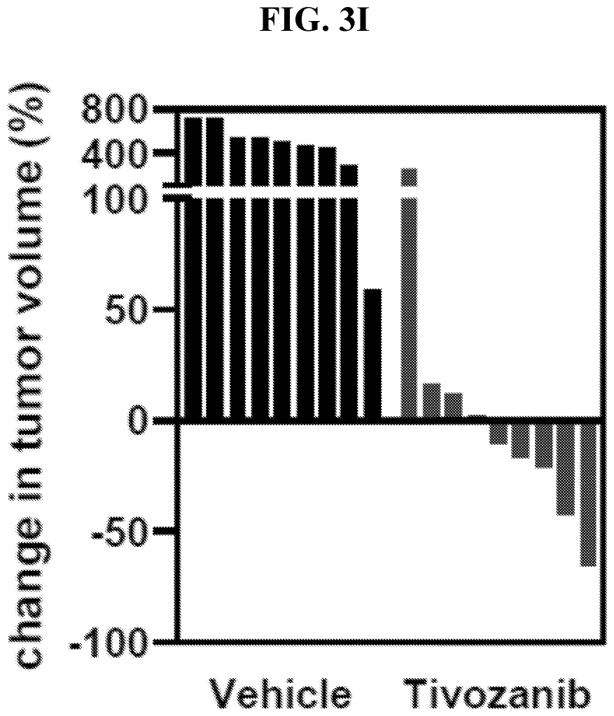

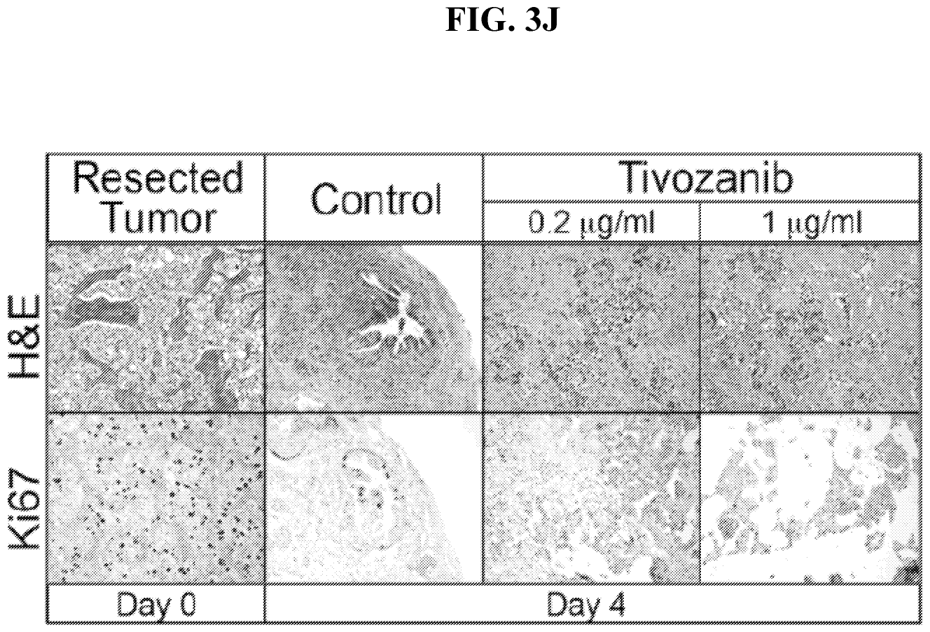

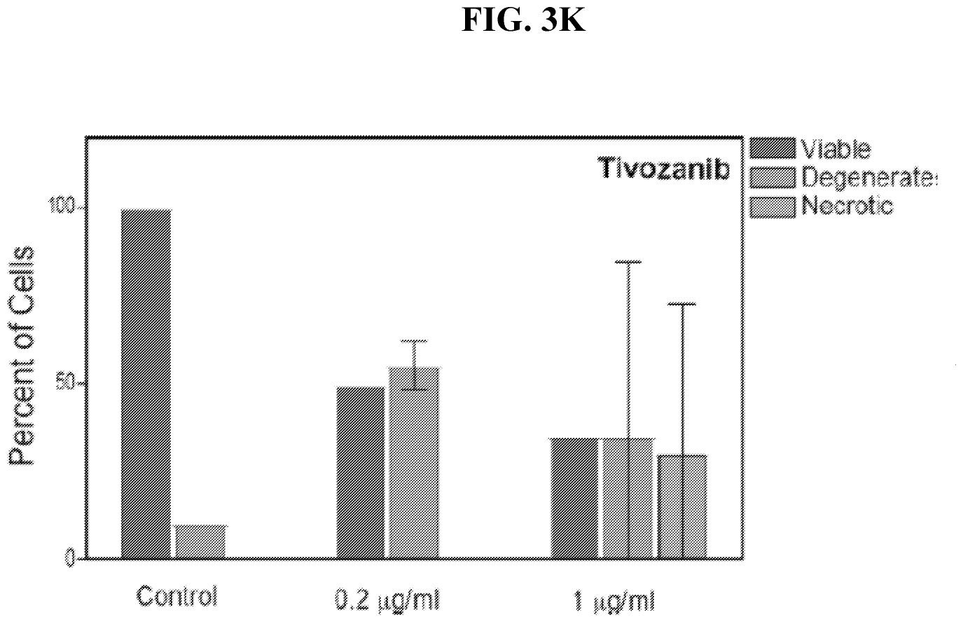

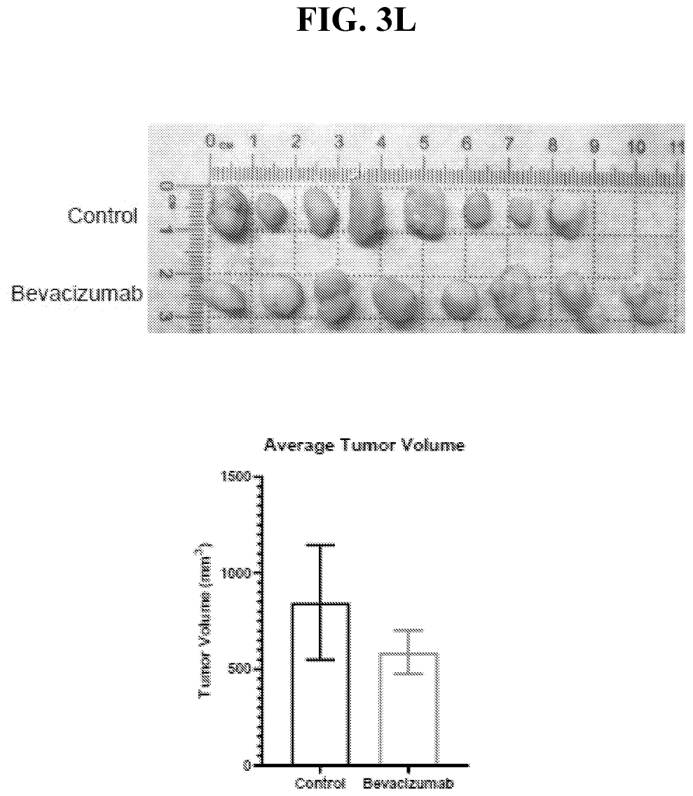

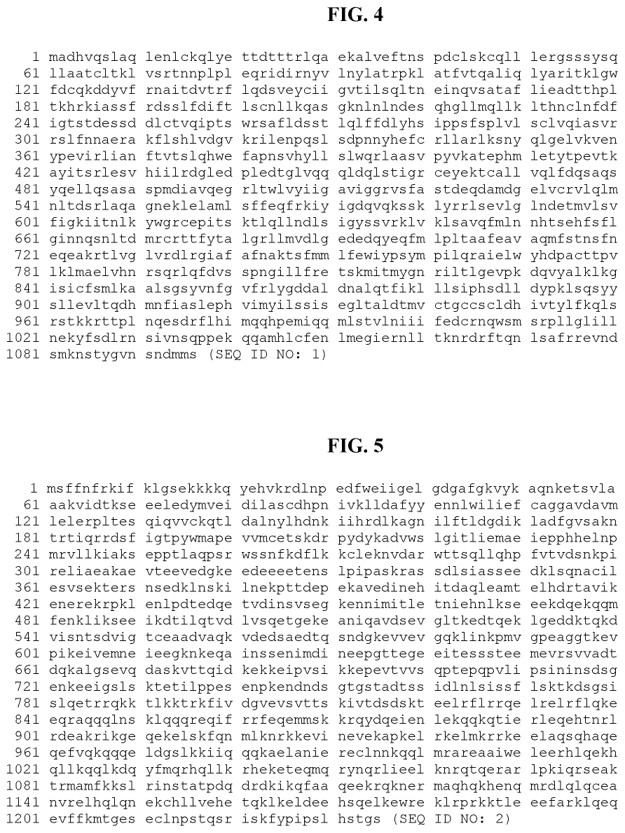

To evaluate the potential therapeutic efficacy of certain small molecules in the treatment of cholangiocarcinoma, a kinome inhibition screen was performed, identifying tivozanib, a pan-VEGFR inhibitor, as a potent inhibitor of SLK (FIG. 3A). Tivozanib inhibited AKT S473 phosphorylation in three cholangiocarcinoma cell lines, WITT, EGI-1, and SNU-1079, (FIG. 3B) and inhibited proliferation of these cells, as shown in the scatter plots of IC50 values in FIGS. 3C-3E.

In a tumor organoid assay, tivozanib abrogated tumor formation in three cholangiocarcinoma cell lines, WITT, EGI-1 and SNU-1070. As shown in FIG. 3F, tumor organoids were fewer and smaller in the tivozanib treated group compared to controls.

In a subcutaneous xenograft model (EGI-1 cell line), tivozanib inhibited tumor growth in vivo. As shown in FIGS. 3G and 3H, tumors treated with tivozanib at 20 mg/kg/day for 28 days were statistically significantly smaller in volume than those treated with control vehicle. The substantial change in tumor volume over the course of tivozanib treatment as compared to control is shown in the waterfall plot in FIG. 3I.

Finally, tivozanib was evaluated in the ex vivo tumor SMART platform described in International Patent Application No. WO2021/183527 which is incorporated by reference herein for all purposes. A liver metastasis from a patient with XPO7-expressing cholangiocarcinoma was evaluated using H&E staining and Ki67 staining to contrast treatment with control versus tivozanib. As shown in FIGS. 3J and 3K, treatment with 0.2 μg/mL tivozanib and 1 μg/mL tivozanib resulted in fewer viable cells compared to the control as well statistically significant increased cell degeneration at both dose levels, as well as statistically significant levels of cell death at the higher dose level (FIG. 3J and FIG. 3K).

To confirm that the tumoricidal effects of tivozanib were not due to its activity as a VEGF inhibitor, but could be attributed to action on SLK, the antitumor effects of bevacizumab, an anti-VEGF antibody, on EGI-1 cholangiocarcinoma xenografts were evaluated. The results following 28 days of treatment are shown in FIG. 3L. While the average volume of tumors treated with bevacizumab was statistically significantly smaller than the control group, the decrease was not as significant as the decrease shown in FIG. 3H where a similar model was used with tivozanib. If the decrease in tumor size with tivozanib was due solely to its activity as a VEGF inhibitor, bevacizumab should have produced similar results. However, it did not, indicating that tivozanib is involved in inhibition of SLK/XPO7 and its effects are not doe to VEGF inhibition alone.

Taken together, these results support evaluation of tivozanib as a treatment option for cholangiocarcinoma in human subjects.

### Example 4: Phase I/II Clinical Trial to Assess Safety and Efficacy of Tivozanib in Human Cholangiocarcinoma Patients

In light of the data presented in Examples 1-3, above, a Phase I clinical study was warranted to determine the safety of tivozanib in treating cholangiocarcinoma and to establish a recommended Phase II dose (RP2D) for tivozanib. A Phase I/II study is planned according to the protocol that follows.

### Objectives

### Primary Objectives

Phase I: To determine safety and establish the recommended Phase II dose (RP2D) of Tivozanib in patients with cholangiocarcinoma who were previously treated with first-line chemotherapy; and
Phase II: To determine the overall response rate (Response Evaluation Criteria in Solid Tumors (RECIST)) of Tivozanib in patients with cholangiocarcinoma who were previously treated with first-line therapy.

### Secondary Objectives

Evaluate disease control response (DCR-complete response (CR), partial response (PR), and stable disease (SD)) in patients with cholangiocarcinoma treated with Tivozanib; and
Evaluate overall survival (OS) in patients with cholangiocarcinoma treated with Tivozanib. PFS and OS will be evaluated by Kaplan-Meier analysis.

### Exploratory Objective

Correlate responses to Tivozanib treatment.

### Eligibility Assessment and Enrollment

### Eligibility Criteria

Patients must meet at least the following inclusion criteria with respect to cholangiocarcinoma and may be subject to other inclusion criteria depending on other pre-existing disease states.
Patients with histologically or cytologically confirmed cholangiocarcinoma. Archival tumor sample may be used but if archival tissue is not available or is not adequate, tissue biopsy will be required;
Patients must have cholangiocarcinoma that is not amenable to resection;
Patients must have had prior treatment with first line chemotherapy;
Disease must be measurable by RECIST criteria Version 1.1;
Age ≥18 years. Because no dosing or adverse event data are currently available on the use of Tivozanib in subjects <18 years of age, children are excluded from this study, but may be eligible for future pediatric trials;
ECOG performance status≤1;

Patients will be excluded if they meet the following criteria and may be subject to exclusion under other criteria depending on other pre-existing disease states, or previous surgery.
Chemotherapy, small molecule or radiation therapy within 3 weeks prior to administration of first dose of study drug;
Prior treatment with Tivozanib;

### Screening Evaluation

Patients will be subject to screening based on disease history including diagnosis, treatment (e.g., systemic treatments, radiation and surgeries), disease status, and significant prior/ongoing side effects and symptoms. A complete medical history will be taken and complete physical examination given. Laboratory evaluations including blood testing and urinalysis will be undertaken. CT and MRI images of the abdomen and liver will be obtained.

### Treatment Assignment Procedures

Table 2 describes the patient cohorts that will be assigned into the phases of the study. Table 3 describes the trial arms in the various phases of the study.

TABLE 2
Participant cohorts

| Number | Name | Description |
|---|---|---|
| 1 | Phase I | Subjects with unresectable |
| | | cholangiocarcinoma (CCA) who were |
| | | previously treated with first-line |
| | | chemotherapy to establish the recommended |
| | | Phase II dose (RP2D) of Tivozanib. |
| 2 | Phase II | Subjects with unresectable cholangiocarcinoma |
| | | (CCA) who were previously treated with first- |
| | | line chemotherapy enrolled at the RP2D of Tivozanib. |

TABLE 3
Trial arms

| Number | Name | Description |
|---|---|---|
| 1 | Phase I | Tivozanib, orally (P.O.) administered |
| | | daily at 1.0 mg (given on Days |
| | | 1-21 of every 28-day cycle) with intra- |
| | | patient escalation to 1.5 mg daily (given on |
| | | Days 1-21 of every 28-day cycle) and possible dose |
| | | de-escalation to 1.0 mg every other day |
| | | (without interruption for a 28-day cycle) |
| | | if needed to determine RP2D. |
| 2 | Phase II | Tivozanib at the RP2D established in Phase I. |

### Arm Assignment

Patients in Cohort 1 will be directly assigned to Arm 1. Once the dose and administration frequency are established, subsequent patients will be enrolled into Cohort 2 and directly assigned to Arm 2. Of note, patients treated at the RP2D in Arm 1 will be transferred to Arm 2 for efficacy determination, so as to minimize the accrual ceiling.

### Baseline Evaluation

An interim medical history and physical examination, as well as CT and MRI images will be taken within 28 days prior to the first dose of Tivozanib. Laboratory Evaluations are required within 14 days prior to the first dose of Tivozanib.

### Study Implementation

### Study Design

This is a Phase I/II trial to evaluate the safety, tolerability and clinical response rate of Tivozanib treatment for patients with non-resectable or metastatic cholangiocarcinoma (CCA) who were previously treated with first line chemotherapy. The trial will begin in Phase I with a two-dose level, intra-patient dose escalation, and a possible dose de-escalation phase to determine safety and to establish the recommended Phase II dose (RP2D) of Tivozanib. The starting dose (DL1) of Tivozanib is 1 mg taken once a day, and the second dose level is 1.5 mg taken once a day (DL2, the desired Phase II dose). Each cycle of treatment is 28 days, with Tivozanib taken once daily for 3 weeks (Days 1-21) followed by one week with no Tivozanib (Days 22-28). The first 3 patients in Phase I will start at DL1 for the first cycle and escalate to DL2 for their second cycle if there are no dose-limiting toxicities. If the DL2 dose is found to be unsafe, dose de-escalation will occur, back to dose level DL1. If the DL1 dose if found to be unsafe, dose de-escalation will occur, with Tivozanib administered at a dose of 1.0 mg taken every other day (DL-1) without interruption for the 28-day cycle. All patients in the Phase I cohort (Cohort 1) must complete two 28-day cycles of treatment before direct enrollment into the Phase II cohort is initiated.

Following Phase I, the efficacy of Tivozanib will be assessed in a Simon two-stage Phase II clinical trial design that treats a minimum of 12 patients (including the 6 patients from the Phase I portion) or maximum of 16 evaluable patients at a dose determined during Phase I.

To allow for enrollment in the Phase I (Cohort 1), Phase II cohort (Cohort 2), and to account for any in-evaluable patients and screen failures, the accrual ceiling will be set at 30 patients.

Patients will be eligible for continued treatment until toxicity (i.e., dose limiting toxicity or toxicity requiring discontinuation) or progressive disease, with treatment evaluation occurring every 8 weeks.

### Dose Limiting Toxicity

A dose-limiting toxicity (DLT) is defined as any treatment-emergent and related severe toxicity (Grade≥3) occurring during the DLT observation time, defined as Cycle 1 (and Cycle 2 with intra-patient dose escalation only), deemed possibly, probably, or definitely related to Tivozanib, with 2 exceptions:
Any grade of vitiligo, alopecia; and
Grade 3 controllable hypertension.

The following toxicities and conditions will be recorded; however, they will not be considered to be DLTs:
Bilirubin <6×ULN if resolved to <3 or ≤2×the baseline in 7 days after first Tivozanib treatment; and
AST/ALT≤20×ULN if resolved to ≤10×ULN in 7 days after first Tivozanib treatment.

Occurrence of any DLT-defining toxicity after Cycle 1 (and after Cycle 2 with intra-patient dose escalation only) will lead to dose modification; however, it will not be deemed a DLT for purposes of dose escalation.

### Dose Escalation—Phase I

Dose escalation (or de-escalation if needed) will proceed according to the following schedule (Tables 3 and 4).

TABLE 4
Dose escalation schedule

| Dose Level | Tivozanib dosing regimen |
|---|---|
| Level 2 (DL2) | 1.0 mg P.O. Daily, Days 1-21 |
| *Level 1 (DL1) | 1.0 mg P.O. Daily, Days 1-21 |
| Level −1 (DL −1) | 1.0 mg P.O., Every Other Day, |
| | Days 1-28 |

*Starting level

TABLE 5
Dose escalation guidelines

| Number of patients with DLT at a given | |
|---|---|
| dose level | Escalation decision rule |
| 0 out of 3 for DL1 after Cycle 1 | Enter the same 3 patients at next dose level |
| | (DL2) starting with Cycle 2. |
| 0 out of 3 for DL2 after Cycle 2 | Enroll 3 more patients at DL2. If 0 or |
| | 1 of these 3 patients experience DLT at DL2, |
| | DL2 is the Phase II dose. If 2 or more patients |
| | experience a DLT at DL2, DL2 is not the |
| | Phase II dose. |
| | Then: |
| | Enroll 3 more patients at DL1. If 0 or |
| | 1 patient experiences a DLT at DL1, DL1 is |
| | the Phase II dose. If 2 or more patients |
| | experience a DLT at DL1 (≥2/6 total), de- |
| | escalate and enroll next 3 patients at DL −1 |
| | (see below). |
| | Enter the 2 current patients without a DLT at |
| | next dose level (DL2) starting with Cycle 2. |
| | If 1 or more of these patients experience a |
| 1 out of 3 at DL1 after Cycle 1 | DLT at DL2, DL2 is not the Phase II dose. If |
| | 0 of these patients experience a DLT at DL2, |
| | then: |
| | Enroll 3 more patients at DL1. If 0 of |
| | these 3 additional patients experience DLT at |
| | DL1 (≤1/6 total) after Cycle 1, enter the same |
| | 3 patients at DL2. If 0 of these patients |
| | experience a DLT at DL2, DL2 is the Phase II |
| | dose. If 1 or more of these 3 patients |
| | experience a DLT a DL2, DL1 is the Phase II |
| | dose. |
| | If 1 or more of the additional 3 |
| | patients experience DLT at DL1 (≥2/6 total) |
| | after Cycle 1, de-escalate and enroll next 3 |
| | patients at DL −1 (see below). |
| 2 or more out of 3 at DL1 after Cycle 1 | DL1 is not the Phase II dose. De-escalate and |
| | enroll next 3 patients at DL −1 (see below). |
| 1 out of 3 at DL2 after Cycle 2 | Enroll 3 more patients at DL2. If 0 of |
| | these 3 additional patients experience DLT at |
| | DL2 (≤1/6 total), DL2 is the Phase II dose. If |
| | 1 or more of the additional 3 patients |
| | experience DLT at DL2 (≥2/6 total), DL2 is |
| | not the Phase II dose. |
| | Enroll 3 more patients at DL1. If ≤1/6 |
| | total patients, DL1 is the Phase II dose. |
| | If ≥2/6 total patients experience a DLT at DL1, |
| | deescalate and enroll next 3 patients at DL −1 |
| | (see below). |
| 2 or more out of 3 at DL2 after Cycle 2 | DL2 is not the Phase II dose. Enroll 3 |
| | more patients at DL1. If ≤1/6 total patients, |
| | DL1 is the Phase II dose. If ≥2/6 total |
| | patients experience a DLT at DL1, deescalate |
| | and enroll next 3 patients at DL −1 (see |
| | below). |
| 0 out of 3 at DL −1 | Enroll 3 more patients at this dose |
| | level. If 0 or 1 of these 3 patients experience |
| | DLT at DL −1 (<1/6), DL −1 is the Phase II |
| | dose. |
| 1 out of 3 at DL −1 | Enroll 3 more patients at this dose |
| | level. If 0 of these 3 patients experience DLT |
| | at DL −1 |
| | (≤1/6 total), all subsequent patients will be |
| | treated at DL −1. DL −1 is the Phase 2 dose. |
| | If 1 of these 3 patients experience |
| | DLT (≥2/6), trial must be halted, pending |
| | consultation with drug manufacturer. |
| 2 or more out of 3 at DL −1 | Trial must be halted, pending consultation |
| | with drug manufacturer. |

### Drug Administration

Tivozanib will be supplied as 1.0 mg or 1.5 mg capsules for daily (or every other day) oral (P.O.) administration on Days 1-21 of a cycle (or every other day of a 28-day cycle). Each cycle is 28 days (4 weeks). Cycles may be delayed due to scheduling or other administrative reasons (i.e., reasons other than toxicity/dose management as defined below) for up to 7 days.

Tivozanib should be taken at approximately the same time every day (24 hours apart, +/−8 hours) continuously for Days 1-21 with 1 week off medication (except for those patients assigned to DL-1, where Tivozanib should be taken every other day, around 48 hours apart for a 28-day cycle).

Tivozanib can be taken with or without food. If the patient vomits or misses a dose of Tivozanib, the patient will be instructed to take the next dose at its scheduled time.

### Dose Modification

### Treatment Modifications for Adverse Reactions

All treatment modifications should be discussed with the senior clinical investigators on the study. Summary of dose holding/interruptions will follow the guidelines outlined in Tables 5 and 6, below. Dose reduction will be recommended for patients with greater than or equal to Grade 3 adverse events except for hypertension, which must be treated with anti-hypertensive drugs prior to consideration for dose reduction. Tivozanib should be held for Grade 4 adverse events. Any serum bilirubin elevation greater than 2×ULN with concomitant elevation of a serum transaminase greater than 3×the ULN will result in discontinuation of treatment. Additionally, if a patient develops a DVT while on study that requires treatment with anticoagulants, irrespective of the type of agent used, Tivozanib will be discontinued and the patient will be taken off treatment. Due to the high incidence of patients that experience hypertension on Tivozanib, blood pressure should be well controlled prior to initiating therapy. While on therapy, all patients will keep a diary and record their blood pressure twice daily. Patients with systolic blood pressure readings of ≥140 mm Hg and/or diastolic blood pressure readings of ≥90 mm Hg will have the readings confirmed by a health care provider. If blood pressure readings are confirmed, patients will be treated as needed with initiation or optimization of antihypertensive therapy according to standard medical practice. In the case of persistent hypertension despite use of antihypertensive medications, the Tivozanib dose will be reduced. Tivozanib will be discontinued if hypertension is severe and persistent despite antihypertensive therapy and dose reduction. Discontinuation will be considered if there is evidence of hypertensive crisis defined as systolic pressures of ≥180 mm Hg or diastolic pressure of ≥120 mm Hg. If Tivozanib is interrupted, subjects receiving antihypertensive medications will be monitored for hypotension. All grading scales in the following tables are according to the revised NCI Common Terminology Criteria for Adverse Events (CTCAE) version 5.0.

TABLE 6
Dose modifications based on blood pressure

| Criteria for treatment or dose modification | Treatment or dose modification |
|---|---|
| ≥140 mm Hg (systolic) and <180 mm | Confirmation of BP reading by a health care provider; |
| Hg OR ≥90 mm Hg (diastolic) and <120 | Initiation or optimization of |
| mm Hg | antihypertensive therapy |
| ≥140 mm Hg (systolic) and <180 mm | Dose reduction |
| Hg OR ≥90 mm Hg (diastolic) and <120 | |
| mm Hg despite optimized | |
| antihypertensive therapy | |
| Severe and persistent BP of ≥140 mm Hg | Discontinuation of therapy |
| (systolic) and <180 mm Hg OR ≥90 mm | |
| Hg (diastolic) and <120 mm Hg despite | |
| dose reduction | |
| One-time confirmed BP reading of ≥180 | Discontinuation of therapy will be considered |
| mm Hg (systolic) OR ≥120 mm Hg | |
| (diastolic) | |

TABLE 7
General dose modifications

| General adverse events | Action |
|---|---|
| Non-hematological, Grade 1 or 2 | Continue Tivozanib therapy at full dose |
| | prescribed. Apply maximum supportive |
| | care recommendations. If prolonged |
| | duration of Grade 2 adverse event (≥7 |
| | days) is affecting quality of life, consider |
| | dose reduction* or discontinuation of the |
| | drug. |
| | For Grade 2 proteinuria, hold Tivozanib |
| | therapy until recovery to Grade ≤1. |
| Non-hematological, Grade 3 or 4 | Apply maximum supportive care |
| | recommendations. Hold Tivozanib |
| | therapy until recovery to Grade ≤1 (up to |
| | 14 days). |
| | For Grade 4 proteinuria (nephrotic |
| | syndrome) Tivozanib must be |
| | discontinued. |
| | If recurrence of adverse event after drug |
| | hold/interruptions is observed, and |
| | maximum supportive care measures |
| | applied, hold drug once again until |
| | recovery to Grade ≤1 (up to 14 days). If |
| | no recovery, consider discontinuation of |
| | the drug. |
| Non-hematological, Grade 3 or 4 adverse | Action (discontinue or resume Tivozanib |
| events NOT resolved to Grade ≤1 within a | therapy) in individual cases after discussions |
| maximum of 2 weeks from last planned | with the Sponsor. |
| administration | |

*If the RP2D is 1.5 mg daily for Days 1-21, dose will be de-escalated to 1.0 mg daily for Days 1-21. If the RP2D is 1.0 mg daily for Days 1-21, dose will be de-escalated to 1.0 mg every other day for Days 1-28. If the RP2D is 1.0 mg every other day for Days 1-28, Tivozanib will be discontinued and the patient will be taken off study.

on-Study Evaluations

After Cycle 1, pre-dose assessments may be performed up to 3 days prior to a cycle except where otherwise noted. The results from all procedures/tests must be reviewed prior to initiation of each cycle of treatment for consideration of dose modifications and delay of therapy.

Treatment with Tivozanib will continue until disease progression, unacceptable treatment-related toxicity or other reasons. Participants will be expected to record their blood pressure twice a day at home for all 28-days of each cycle of treatment.

### Post-Treatment Evaluations

An End of Treatment Visit will be performed approximately 30 days after the last dose of protocol treatment. If a subject initiates a new anti-cancer therapy within 30 days after the last dose of trial treatment, the 30-day End of Treatment Visit must occur before the first dose of the new therapy, if possible.

Long term follow-up will occur as follows: at 6 months post-End of Treatment Visit, then at 12 months after End of Treatment Visit, and then annually.

Criteria for Removal from Protocol Therapy and Off-Study Criteria

Prior to removal from study, effort must be made to have all patients complete a safety visit approximately 30 days following the last dose of study therapy. Additional safety visits and follow-up will continue.

Criteria for Removal from Protocol Therapy
Confirmed disease progression;
Intercurrent illness that prevents further administration of treatment;
Unacceptable toxicities or toxicities that require treatment to be stopped;
New development of serum bilirubin greater than 2×ULN in conjunction with serum transaminases greater than 3×ULN, and not treatable with a biliary stent;
Development of a DVT that requires treatment with anticoagulants, irrespective of the type of agent used;
Subject's request to withdraw from protocol therapy;
Investigator's decision to withdraw the patient;
Subject's non-compliance with trial treatment or procedure requirements that requires removal in the opinion of the investigator;
Pregnancy;
The drug manufacturer can no longer provide the study agent; and/or
Study is cancelled for any reason.

### Off-Study Criteria

Subject requests to be withdrawn from study;
Subject is lost to follow-up;
Death; and/or
Study is cancelled for any reason.

### Concomitant Medications/Measures

All treatments that the investigator considers necessary for a subject's welfare may be administered at the discretion of the investigator in keeping with the community standards of medical care. All concomitant medication will be recorded on the case report form (CRF) including all prescription, over-the-counter (OTC), herbal supplements, and IV medications. If changes occur during the trial period, documentation of drug dosage, frequency, route, and date may also be included on the CRF.

All concomitant medications received within 28 days before the first dose of trial treatment and 30 days after the last dose of trial treatment should be recorded. Co-administration of CYP3A4 inhibitors will be avoided; however, they can be administered if it is determined by the principal investigator that the benefit would outweigh the risk.

If a patient develops a DVT while on study that requires treatment with anticoagulants, irrespective of the type of agent used, Tivozanib will be discontinued and the patient will be taken off treatment.

### Biospecimen Collection

TABLE 8
Specimen collection table

| | Volume | Type of | Collection points | Mandatory |
|---|---|---|---|---|
| Test/Assay | (approx.) | tube | (±48 hrs) | Collection |
| Circulating | Blood, 20 | CellSave | Baseline, and Treatment | Yes |
| Tumor Cell | mL | Preservation | Eval (prior to imaging) | |
| Analysis | | Tubes | | |
| Biomarker | Blood, | EDTA, | Baseline, and Treatment | Yes |
| Studies | 10 mL | Lavender | Eval (prior to imaging) | |
| | | top | | |
| Exosome | Blood, 10 | EDTA, | Baseline, and Treatment | Yes |
| Analyses | mL | Lavender | Eval (prior to imaging) | |
| | | top | | |
| Tumor Biopsy for | Core | Formalin | Baseline*, and Time of | No |
| IHC | Needle | and Frozen | RECIST Response or | |
| | | | Progression | |
| Tumor Biopsy for | Core | Formalin | Baseline*, and Time of | No |
| Next Gen | Needle | and Frozen | RECIST Response or | |
| Sequencing | | | Progression | |

*Tissue biopsy is mandatory at screening if adequate archival tissue is not available to confirm diagnosis. An additional biopsy is not needed at baseline if adequate archival tissue remains for research studies.

### Sample Collection and Processing

### Tissue Samples

Tissue samples will be collected from subjects for immunohistochemistry (IHC) and next-generation sequencing evaluations for XPO7, SLK.

### Sample Storage, Tracking, and Disposition

Samples are stored in barcoded boxes in a locked freezer at either −20° C. or −80° C. according to stability requirements.

### Samples for Genetic/Genomic Analysis

The research correlates for this study may include DNA/RNA sequencing of tumors.

### Response Criteria

### Response Assessments

For the purposes of this study, patients should be re-evaluated for response every 8 weeks. In addition to a baseline scan, confirmatory scans should also be obtained 8 weeks (not less than 4) weeks following initial documentation of objective response. Response and progression will be evaluated in this study using the new international criteria proposed by the revised Response Evaluation Criteria in Solid Tumors (RECIST) guideline (version 1.1). Changes in the largest diameter (unidimensional measurement) of the tumor lesions and the shortest diameter in the case of malignant lymph nodes are used in the RECIST criteria. CT or MRI will be used for response criteria.

### Disease Parameters

Measurable disease: Measurable lesions are defined as those that can be accurately measured in at least one dimension (longest diameter to be recorded) as:
   By chest x-ray: ≥20 mm;
   By CT/MR scan:
      Scan slice thickness 5 mm or under as >10 mm with CT scan;
      Scan slice thickness >5 mm: double the slice thickness
   With calipers on clinical exam: >10 mm.
All tumor measurements must be recorded in millimeters (or decimal fractions of centimeters).
Malignant lymph nodes: To be considered pathologically enlarged and measurable, a lymph node must be >15 mm in short axis when assessed by CT scan (CT scan slice thickness recommended to be no greater than 5 mm). At baseline and in follow-up, only the short axis will be measured and followed.
Non-measurable disease: All other lesions (or sites of disease), including small lesions (longest diameter <10 mm or pathological lymph nodes with ≥10 to <15 mm short axis), are considered non-measurable disease. Bone lesions, leptomeningeal disease, ascites, pleural/pericardial effusions, lymphangitis cutis/pulmonitis, inflammatory breast disease, and abdominal masses (not followed by CT or MRI), are considered as non-measurable. Note: Cystic lesions that meet the criteria for radiographically defined simple cysts should not be considered as malignant lesions (neither measurable nor non-measurable) since they are, by definition, simple cysts. ‘Cystic lesions’ thought to represent cystic metastases can be considered as measurable lesions, if they meet the definition of measurability described above. However, if non-cystic lesions are present in the same patient, these are preferred for selection as target lesions.
Target lesions: All measurable lesions up to a maximum of 2 lesions per organ and 5 lesions in total, representative of all involved organs, should be identified as target lesions and recorded and measured at baseline. Target lesions should be selected on the basis of their size (lesions with the longest diameter), be representative of all involved organs, but in addition should be those that lend themselves to reproducible repeated measurements. It may be the case that, on occasion, the largest lesion does not lend itself to reproducible measurement in which circumstance the next largest lesion which can be measured reproducibly should be selected. A sum of the diameters (longest for non-nodal lesions, short axis for nodal lesions) for all target lesions will be calculated and reported as the baseline sum diameters. If lymph nodes are to be included in the sum, then only the short axis is added into the sum. The baseline sum diameters will be used as reference to further characterize any objective tumor regression in the measurable dimension of the disease.
Non-target lesions: All other lesions (or sites of disease) including any measurable lesions over and above the 5 target lesions should be identified as non-target lesions and should also be recorded at baseline. Measurements of these lesions are not required, but the presence, absence, or in rare cases unequivocal progression of each should be noted throughout follow-up.

### Methods for Evaluation of Measurable Disease

All measurements should be taken and recorded in metric notation using a ruler or calipers. All baseline evaluations should be performed as closely as possible to the beginning of treatment and never more than 4 weeks before the beginning of the treatment. The same method of assessment and the same technique should be used to characterize each identified and reported lesion at baseline and during follow-up. Imaging-based evaluation is preferred to evaluation by clinical examination unless the lesion(s) being followed cannot be imaged but are assessable by clinical exam.

Conventional CT and MRI: This guideline has defined measurability of lesions on CT scan based on the assumption that CT slice thickness is 5 mm or less. If CT scans have slice thickness greater than 5 mm, the minimum size for a measurable lesion should be twice the slice thickness. MRI is also acceptable in certain situations (e.g., for body scans). Use of MRI remains a complex issue. MRI has excellent contrast, spatial, and temporal resolution; however, there are many image acquisition variables involved in MRI, which greatly impact image quality, lesion conspicuity, and measurement. Furthermore, the availability of MRI is variable globally. As with CT, if an MRI is performed, the technical specifications of the scanning sequences used should be optimized for the evaluation of the type and site of disease. Furthermore, as with CT, the modality used at follow-up should be the same as was used at baseline and the lesions should be measured/assessed on the same pulse sequence. It is beyond the scope of the RECIST guidelines to prescribe specific MRI pulse sequence parameters for all scanners, body parts, and diseases. Ideally, the same type of scanner should be used, and the image acquisition protocol should be followed as closely as possible to prior scans. Body scans should be performed with breath-hold scanning techniques, if possible.

PET-CT: At present, the low dose or attenuation correction CT portion of a combined PET-CT is not always of optimal diagnostic CT quality for use with RECIST measurements. However, if the site can document that the CT performed as part of a PET-CT is of identical diagnostic quality to a diagnostic CT (with IV and oral contrast), then the CT portion of the PET-CT can be used for RECIST measurements and can be used interchangeably with conventional CT in accurately measuring cancer lesions over time. Note, however, that the PET portion of the CT introduces additional data which may bias an investigator if it is not routinely or serially performed.

Ultrasound: Ultrasound is not useful in assessment of lesion size and should not be used as a method of measurement. Ultrasound examinations cannot be reproduced in their entirety for independent review at a later date and, because they are operator dependent, it cannot be guaranteed that the same technique and measurements will be taken from one assessment to the next. If new lesions are identified by ultrasound in the course of the study, confirmation by CT or MRI is advised. If there is concern about radiation exposure at CT, MRI may be used instead of CT in selected instances.

Endoscopy, Laparoscopy: The utilization of these techniques for objective tumor evaluation is not advised. However, such techniques may be useful to confirm complete pathological response when biopsies are obtained or to determine relapse in trials where recurrence following complete response (CR) or surgical resection is an endpoint.

Tumor markers: Tumor markers alone cannot be used to assess response. If markers are initially above the upper normal limit, they must normalize for a patient to be considered in complete clinical response. Specific guidelines for both CA 125 response (in recurrent ovarian cancer) and PSA response (in recurrent prostate cancer) are well-known. In addition, the Gynecologic Cancer Intergroup has developed CA-125 progression criteria which are to be integrated with objective tumor assessment for use in first-line trials in ovarian cancer. CA 19-9 Levels will be drawn with each evaluative imaging.

Cytology, Histology: These techniques can be used to differentiate between partial responses (PR) and complete responses (CR) in rare cases (e.g., residual lesions in tumor types, such as germ cell tumors, where known residual benign tumors can remain). The cytological confirmation of the neoplastic origin of any effusion that appears or worsens during treatment when the measurable tumor has met criteria for response or stable disease is mandatory to differentiate between response or stable disease (an effusion may be a side effect of the treatment) and progressive disease.

### Response Criteria

### Evaluation of Target Lesions

Complete Response (CR): Disappearance of all target lesions. Any pathological lymph nodes (whether target or non-target) must have reduction in short axis to <10 mm.

Partial Response (PR): At least a 30% decrease in the sum of the diameters of target lesions, taken as reference the baseline sum of diameters.

Progressive Disease (PD): At least a 20% increase in the sum of the diameters of target lesions, taken as reference the smallest sum on study (this includes the baseline sum if that is the smallest on study). In addition to the relative increase of 20%, the sum must also demonstrate an absolute increase of at least 5 mm. Note: the appearance of one or more new lesions is also considered as disease progression).

Stable Disease (SD): Neither sufficient shrinkage to qualify for PR nor sufficient increase to qualify for PD, taken as reference the smallest sum of diameters while on study.

### Evaluation of Non-Target Lesions

Complete Response (CR): Disappearance of all non-target lesions and normalization of tumor marker level. All lymph nodes must be non-pathological in size (<10 mm short axis). Note: If tumor markers are initially above the upper normal limit, they must normalize for a patient to be considered in complete clinical response.

Non-CR/Non-PD: Persistence of one or more non-target lesion(s) and/or maintenance of tumor marker level above the normal limits.

Progressive Disease (PD): Appearance of one or more new lesions and/or unequivocal progression of existing non-target lesions. Unequivocal progression should not normally trump target lesion status. It must be representative of overall disease status change, not a single lesion increase. Although a clear progression of “non-target” lesions only is exceptional, the opinion of the treating physician should prevail in such circumstances, and the progression status should be confirmed at a later time by the review panel (or Principal Investigator).

### Evaluation of Best Overall Response

The best overall response is the best response recorded from the start of the treatment until disease progression/recurrence (taken as reference for progressive disease the smallest measurements recorded since the treatment started). The patient's best response assignment will depend on the achievement of both measurement and confirmation criteria.

TABLE 9
For Patients with Measurable Disease (i.e., Target Disease)

| | | | | Best overall |
|---|---|---|---|---|
| | | | | response |
| | | | | when |
| Target | Non-target | New | Overall | confirmation |
| lesions | lesions | lesions | response | is required* |
| CR | CR | No | CR | ≥4 wks. |
| | | | | Confirmation** |
| CR | Non-CR/Non- | No | PR | ≥4 wks. |
| | PD | | | Confirmation** |
| CR | Not evaluated | No | PR | |
| PR | Non-CR/Non- | No | PR | |
| | PD/not | | | |
| | evaluated | | | |
| SD | Non-CR/Non- | No | SD | Documented at |
| | PD/not | | | least once ≥4 |
| | evaluated | | | wks. from |
| | | | | baseline** |
| PD | Any | Yes or No | PD | NBo prior SD, |
| Any | PD*** | Yes or No | PD | PR or CR |
| Any | Any | Yes | PD | |

*See RECIST 1.1 manuscript for further details on what is evidence of a new lesion.
**Only for non-randomized trials with response as primary endpoint.
***In exceptional circumstances, unequivocal progression in non-target lesions may be accepted as disease progression.
Note:
Patients with a global deterioration of health status requiring discontinuation of treatment without objective evidence of disease progression at that time should be reported as “symptomatic deterioration.” Every effort should be made to document the objective progression even after discontinuation of treatment.

TABLE 10
For Patients with Non-Measurable Disease
(i.e., Non-Target Disease)

| Non-target lesions | New lesions | Overall response |
|---|---|---|
| CR | No | CR |
| Non-CR/Non-PD | No | Non-CR/Non-PD* |
| Not all evaluated | No | Not evaluated |
| Unequivocal PD | Yes or No | PD |
| Any | Yes | PD |

*‘Non-CR/non-PD’ is preferred over ‘stable disease’ for non-target disease since SD is increasingly used as an endpoint for assessment of efficacy in some trials so to assign this category when no lesions can be measured is not advised.

### Duration of Response

Duration of overall response: The duration of overall response is measured from the time measurement criteria are met for CR or PR (whichever is first recorded) until the first date that recurrent or progressive disease is objectively documented (taken as reference for progressive disease the smallest measurements recorded since the treatment started). The duration of overall CR is measured from the time measurement criteria are first met for CR until the first date that progressive disease is objectively documented.

Duration of stable disease: Stable disease is measured from the start of the treatment until the criteria for progression are met, taken as reference the smallest measurements recorded since the treatment started, including the baseline measurements.

### Progression-Free Survival

Progression-free survival (PFS) is defined as the duration of time from the date of study enrollment until time of disease relapse, disease progression, or death, whichever occurs first.

### Overall Survival

Overall survival (OS) is defined as the time from the date of study enrollment until time of death from any cause.

### Toxicity Criteria

Adverse Event (AE): Any untoward medical occurrence in a patient or clinical investigation subject administered a pharmaceutical product and which does not necessarily have a causal relationship with this treatment. An AE can therefore be any unfavorable and unintended sign (including an abnormal laboratory finding), symptom, or disease temporally associated with the use of a medicinal (investigational) product, whether or not related to the medicinal (investigational) product.

Serious Adverse Event (SAE): An adverse event or suspected adverse reaction is considered serious if in the view of the investigator or the sponsor, it results in any of the following:
Death;
Life-threatening adverse event;
Inpatient hospitalization or prolongation of existing hospitalization:
   A hospitalization/admission that is pre-planned (i.e., elective or scheduled surgery arranged prior to the start of the study), a planned hospitalization for pre-existing condition, or a procedure required by the protocol, without a serious deterioration in health, is not considered a serious adverse event;
   A hospitalization/admission that is solely driven by non-medical reasons (e.g., hospitalization for patient convenience) is not considered a serious adverse event; and
   Emergency room visits or stays in observation units that do not result in admission to the hospital would not be considered a serious adverse event. The reason for seeking medical care should be evaluated for meeting one of the other serious criteria;
Persistent or significant incapacity or substantial disruption of the ability to conduct normal life functions;
Congenital anomaly/birth defect; and
Important medical events that may not result in death, be life-threatening, or require hospitalization may be considered a serious adverse drug experience when, based upon appropriate medical judgment, they may jeopardize the patient or subject and may require medical or surgical intervention to prevent one of the outcomes listed in this definition.

Life threatening: An adverse event or suspected adverse reaction is considered “life-threatening” if, in the view of either the investigator or sponsor, its occurrence places the patient or subject at immediate risk of death. It does not include an adverse event or suspected adverse reaction that, had it occurred in a more severe form, might have caused death.

The following adverse event (AE) management guidelines are intended to ensure the safety of each patient while on the study. The descriptions and grading scales found in the revised NCI Common Terminology Criteria for Adverse Events (CTCAE) version 5.0 will be utilized for AE.

### Non-Target Lesions New Lesions Overall Response

CR No CR;
Non-CR/non-PD No Non-CR/non-PD*;
Not all evaluated No not evaluated;
Unequivocal PD Yes or No PD; and.
Any Yes PD
‘Non-CR/non-PD’ is preferred over ‘stable disease’ for non-target disease since SD is increasingly used as an endpoint for assessment of efficacy in some trials so to assign this category when no lesions can be measured is not advised reporting. All appropriate treatment areas should have access to a copy of the CTCAE version 5.0. A copy of the CTCAE version 5.0 can be downloaded from the CTEP web site (ctep.cancer.gov/protocolDevelopment/electronic_applications/ctc.htm).

### Statistical Considerations

### Statistical Hypothesis

Primary Endpoints:
   Phase I: Safety and tolerability and establishment of RP2D; and
   Phase II: Overall response rate by RECIST.
Secondary Endpoints:
   Disease control response (DCR); and
   Overall survival (OS).

### Sample Size Determination

The trial will begin with enrollment in a two dose-level, intra-patient dose escalation and possible dose de-escalation phase to determine safety and to establish the recommended Phase II dose (RP2D) of Tivozanib. In order to complete the dose escalation, between 6 and 12 patients are required. Using a standard 3+3 design, the first 3-6 patients in the dose escalation will receive a dose of 1.0 mg Q.D. (DL1) during Cycle 1.

If 0/3 patients have a dose limiting toxicity at DL1 during Cycle 1, then the same 3 patients will be dose escalated and treated at the intended Phase II dose of 1.5 mg Q.D. (DL2) starting with Cycle 2 and likewise evaluated for dose limiting toxicities.

If 0/3 patients have a dose limiting toxicity at DL2 then an additional 3 patients will be enrolled and treated at the intended Phase II dose of 1.5 mg (DL2) and likewise evaluated for dose limiting toxicities.

If 0 or 1/6 patients experience DLT at DL2 then then the trial will proceed to Phase II portion with a RP2D of at 1.5 mg (DL2). If 1/3 patients experience a dose limiting toxicity at DL1 after Cycle 1, then the 2 patients without a DLT will be escalated to DL2.

If 1 or more of these 2 patients experiences a DLT at DL2, DL2 will not the RP2D. If 0/2 patients experience a DLT at DL2, then an additional 3 patients will be enrolled at DL1. If 0 of these 3 additional patients experience DLT at DL1 (≤1/6 total) after Cycle 1, the same 3 patients will be enrolled at DL2.

If 0/3 patients experience a DLT at DL2 (≤1/6 patients total), DL2 will be the RP2D. If 1 or more of the additional 3 patients experience DLT at DL2 (>2/6 total), DL1 will be the RP2D.

If 1 or more of these additional 3 patients experience a DLT at DL1 (≥2/6 total), those patients will be de-escalated and the next 3 patients enrolled at 1.0 mg every other day (DL-1).

If ≤1/6 patients experience a DLT at DL-1, then the remaining patients will be enrolled at this dose level in the Phase II portion of the trial.

Should there be 2 or more DLTs at DL-1, then no further patients will be enrolled.

In summary, the highest dose level at which 0-1/6 patients have a dose limiting toxicity will be expanded as described above for the Phase II portion of the study to include the 6 patients treated at the RP2D in Phase I.

Following the Phase I portion, this trial will be conducted using a Simon minimax two-stage Phase II trial design, to rule out an unacceptably low response rate (CR+PR) of 5% (p0=0.05) in favor of an improved response rate of 25% (p1=0.25) in order to determine if Tivozanib would more than double the previously reported ORR of 7.7% among patients with cholangiocarcinoma who have received a second line regimen in Phase II studies following first line chemotherapy.

With alpha=0.10 (probability of accepting a poor treatment=0.10) and beta=0.20 (probability of rejecting a good treatment=0.20), the first stage will enroll 12 evaluable patients, including the 6 patients from the dose escalation portion treated at RP2D, and if 0 of the 12 has a response, then no further patients will be accrued.

If 1 or more of the first 12 patients has a response, then accrual will continue until a total of 16 evaluable patients have been treated. As it may take up to several months to determine if a patient has experienced a response, a temporary pause in the accrual may be necessary to ensure that enrollment to the second stage is warranted.

If there are only 1-2 patients with a response out of 16 patients, this would be an uninterestingly low response rate.

If there were 3 or more of 16 (18.8%) who experienced a response, this would be sufficiently interesting to warrant further study in later trials. Under the null hypothesis (5% response rate), the probability of early termination is 54.0%.

The trial will plan for up to 12 patients during the Phase I dose escalation/de-escalation portion to account for DL1 and either escalation to DL2 or de-escalation to DL-1, although 6 of these patients will be included in Phase II. As such, no more than 3+16=19 are expected to be enrolled but it is possible that up to 6+16=22 evaluable patients may be necessary; it is expected that they can be accrued within 2 years. To allow for a small number of patients who may be unevaluable for response, and to allow for screen failures, the accrual ceiling will be set at 30 patients.

### Population for Analysis

### Evaluable for Toxicity

All patients will be evaluable for toxicity from the time of their first treatment with Tivozanib.

### Evaluable for Objective Response

Only those patients who have measurable disease present at baseline, have received at least one cycle of therapy, and have had their disease re-evaluated will be considered evaluable for response. Note: Patients who exhibit objective disease progression prior to the end of Cycle 1 will also be considered evaluable.

### Evaluable Non-Target Disease Response

Patients who have lesions present at baseline that are evaluable but do not meet the definitions of measurable disease, have received at least one cycle of therapy, and have had their disease re-evaluated will be considered evaluable for non-target disease. The response assessment is based on the presence, absence, or unequivocal progression of the lesions.

### Statistical Analyses

### General Approach

DLTs will be counted and reported on all patients in Phase I as the expansion into Phase II. In Phase II, the clinical response rate (CR+PR) will be determined and reported.

### Analysis of the Primary Endpoints

The DLTs will be counted and reported. In Phase II, the clinical response rate (CR+PR) will be determined and reported along with a 95% confidence interval.

### Analysis of the Secondary Endpoints

DCR (CR plus PR plus SD) will be determined and reported along with 95% confidence intervals. PFS and OS will be determined using Kaplan-Meier estimates. Wherever possible, duration of DCR will be reported.

### Safety Analyses

The type, grade, and frequency of toxicities will be reported. During the Phase I portion DLTs will be determined and reported at each dose level.

### Baseline Descriptive Statistics

Standard descriptive statistics of patients will be provided according to phase of trial on which the patients were enrolled, as well as separately for all patients considered to be part of the Phase II portion.

### Planned Interim Analyses

As indicated by the Simon 2-stage design, after 12 evaluable patients have been treated during the Phase II portion, there will be an interim analysis to determine if accrual should proceed to the second stage.

### Exploratory Analyses

As an exploratory analysis, after the trial has ended accrual, patients with and without histologically or cytologically confirmed XPO7-expressing cholangiocarcinoma will be identified, and response rates will be reported separately by XPO7 category, along with 95% confidence intervals on each.

### Pharmaceutical Information

### Formulation and Preparation

Tivozanib drug product is an immediate release, solid oral, hard gelatin capsule filled with a blend of excipients and Tivozanib drug substance. The drug substance is the monohydrate form of the hydrochloride salt of Tivozanib. The clinical drug product is provided in 2 strengths: 1.0 mg and 1.5 mg. A size “4” white, opaque, hard gelatin capsule is used for both strengths; additionally size 4 dark blue (1.0 mg) or bright yellow (1.5 mg) capsules may be used. Bulk Tivozanib capsules are packaged in a primary container closure system consisting of double low-density polyethylene (LDPE) bags. The finished product is packaged in high-density polyethylene (HDPE) bottles with a polypropylene cap with an induction seal.

### Stability and Storage

Tivozanib capsules should be stored at room temperature, 20° C. to 25° C., with excursions permitted between permitted between 15° C. and 30° C. for long-term storage. Tivozanib has sufficient stability studies to allow for short-term excursions during shipping for the range of 2° C. to 40° C.; therefore, no temperature monitoring is required during shipment. Long-term stability studies conducted according to International Council for Harmonization (ICH) guidelines support an expiration dating of 60 months for 1.0 mg and 1.5 mg dose strengths when stored at 20° C. to 25° C. with allowable excursions permitted up to 15° C. to 30° C. Although it is approved for use in treatment of patients with certain relapsed or refractory advanced renal cell carcinoma (RCC), tivozanib is an investigational new drug with respect to bile duct cancers. Accordingly, recommendations and applicable regulations for handling and disposal of orally administered investigational drugs should be followed. Unopened vials must be stored as directed.

### CONCLUSIONS

Using the methods of the present invention, the inventors believe that improved results may be achieved in treatment of bile duct cancers, including cholangiocarcinoma. While these cancers can be difficult to treat, it is believed that targeting expression of the XPO7/SLK pathway with tivozanib can be used to achieve demonstrably beneficial results, such as response according to RESCIST criteria in certain patient populations, who otherwise have a poor prognosis with such cancers.

### INCORPORATION BY REFERENCE

The entire disclosure of each of the patent documents and scientific articles cited herein is incorporated by reference for all purposes.

### EQUIVALENTS

The invention can be embodied in other specific forms with departing from the essential characteristics thereof. The foregoing embodiments therefore are to be considered illustrative rather than limiting on the invention described herein. The scope of the invention is indicated by the appended claims rather than by the foregoing description, and all changes that come within the meaning and range of equivalency of the claims are intended to be embraced therein.

1. A method of treating bile duct cancer in a subject in need thereof comprising:
administering to a subject identified as having bile duct cancer that expresses exportin 7 (XPO7) and/or STE-20 like kinase (SLK), an effective amount of tivozanib, thereby treating the bile duct cancer.

2. (canceled)

3. The method of claim 1, wherein the bile duct cancer expresses XPO7.

4. The method of claim 1, wherein XPO7 is detected in the cytoplasm of bile duct tumor cells from the subject.

5. The method of claim 1, wherein the bile duct cancer expresses SLK.

6. A method of treating bile duct cancer in a subject in need thereof, comprising: administering an effective amount of tivozanib to the subject, thereby treating the bile duct cancer.

7. A method of identifying a subject having a bile duct cancer who is eligible for treatment with tivozanib comprising:
determining whether the bile duct cancer expresses exportin 7 (XPO7) and/or STE-20 like kinase (SLK), wherein the subject is identified as eligible for treatment with tivozanib if the bile duct cancer expresses XPO7 and/or SLK.

8. The method of claim 7, wherein the subject is not eligible for treatment with tivozanib if the bile duct cancer does not express XPO7 and/or does not express SLK.

9. The method of claim 7, wherein the bile duct cancer expresses XPO7.

10. The method of claim 7, wherein XPO7 is detectable in the cytoplasm of bile duct tumor cells from the subject.

11. The method of claim 7, wherein the bile duct cancer expresses SLK.

12. The method of claim 7, further comprising administering an effective amount of tivozanib to the subject, thereby treating the bile duct cancer.

13. The method of claim 1, wherein XPO7 expression is detected by immunohistochemical analysis of a tissue sample from the bile duct cancer using an anti-XPO7 antibody.

14. A method of inhibiting SLK in a bile duct cancer, comprising administering an effective amount of tivozanib to the bile duct cancer, thereby inhibiting SLK in the cancer or tumor.

15. The method of claim 14, wherein the bile duct cancer is in a human subject.

16. The method of claim 1, wherein the bile duct cancer is cholangiocarcinoma (CCA), an intrahepatic cholangiocarcinoma or an extrahepatic cholangiocarcinoma.

17. (canceled)

18. The method of claim 1, wherein the bile duct cancer has been previously treated with chemotherapy.

19. The method of claim 1, wherein the bile duct cancer was previously treated with a platinum chemotherapy, an antimetabolite, a fluoropyrimidine or 5-fluorouracil (5-FU), an FGFR2 inhibitor, an isocitrate dehydrogenase 1 (IDH1) inhibitor, a checkpoint inhibitor, or radiation.

20. The method of claim 19, wherein the bile duct cancer was previously treated with cisplatin, oxaliplatin, carboplatin, gemcitabine or capecitabine.

21-24. (canceled)

25. The method of claim 19, wherein:
the FGFR2 inhibitor is pemigatinib or infigratinib;
the IDH1 inhibitor is ivosidenib; or
the checkpoint inhibitor is an anti-PD1 inhibitor, an anti-PD-L1 inhibitor, a CTLA-4 inhibitor, pembrolizumab, nivolumab, cemiplumab, atezolizumab, avelumab, durvalumab, ipilimumab, tremelimumab or tisotumab.

26-31. (canceled)

32. The method of claim 1, wherein the bile duct cancer was previously surgically resected and has recurred or metastasized.

33. The method of claim 1, wherein the bile duct cancer is unresectable.

34. The method of claim 1, where the bile duct cancer has not been previously treated by chemotherapy, immunotherapy, radiation, or other non-surgical intervention.

35. The method of claim 1, wherein the effective amount of tivozanib is 0.1 mg to 2.0 mg.

36. The method of claim 1, wherein the effective amount of tivozanib is 1.0 mg to 1.5 mg.

37. The method of claim 1, wherein the tivozanib is tivozanib hydrochloride.

38. The method of claim 1, wherein the effective amount of tivozanib is 0.89 mg to 1.34 mg of tivozanib free base.

39. The method of claim 1, wherein;
the effective amount of tivozanib is a treatment cycle of 1.5 mg tivozanib hydrochloride or 1.34 mg tivozanib free base administered once daily for 21 days followed by 7 days without administration of tivozanib;
the effective amount of tivozanib is a treatment cycle of 1.0 mg tivozanib hydrochloride or 0.89 mg tivozanib free base administered once daily for 21 days followed by 7 days without administration of tivozanib;
the effective amount of tivozanib is a treatment cycle of 0.89 mg tivozanib free base administered every other day for 28 days; or
the effective amount of tivozanib is a treatment cycle of 1.34 mg tivozanib free base administered every other day for 28 days.

40-42. (canceled)

43. The method of claim 39, wherein the treatment cycle is repeated 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more times.

44. The method of claim 39, wherein the treatment cycle is repeated until the bile duct cancer progresses, the subject dies, or the subject experiences an unacceptable toxicity.

45. The method of claim 1, wherein the bile duct cancer is advanced, recurrent, or metastatic bile duct cancer.

46. The method of claim 1, wherein the tivozanib is administered orally.

47. The method of claim 1, wherein the tivozanib is a capsule or tablet.

48. The method of claim 1, wherein the subject is not treated with tivozanib if XPO7 expression is detected in the nuclei of cells of the bile duct cancer, but not in the cytoplasm.
